(19) United States
(12) Patent Application Publication
(10) Pub. No.: US 2008/0119491 A1
(43) Pub. Date: May 22, 2008

(54) AMIDINOHETEROCYCLES AS MODULATORS OF INDOLEAMINE 2,3-DIOXYGENASE

(75) Inventor: Andrew P. Combs, Kennett Square, PA (US)

Correspondence Address: FISH & RICHARDSON PC, P.O. BOX 1022, MINNEAPOLIS, MN, 55440-1022, US

(73) Assignee: INCYTE CORPORATION, Wilmington, DE (US)

(21) Appl. No.: 11/856,996

(22) Filed: Sep. 18, 2007

Related U.S. Application Data
(60) Provisional application No. 60/845,687, filed on Sep. 19, 2006.
(60) Provisional application No. 60/900,827, filed on Feb. 12, 2007.

(51) Int. Cl. A61K 31/4965; C07D 241/20; C07D 213/53; A61K 31/4406; C07C 251/48; A61K 31/155; C07D 237/08; A61K 31/50; C12N 9/00; A61P 35/00; A61P 25/00; A61P 31/12

(52) U.S. Cl. 514/255.06; 544/336; 546/332; 514/357; 564/229; 514/633; 544/224; 514/247; 435/183

(57) ABSTRACT

The present invention is directed to amidino heterocyclic compounds which are modulators of indoleamine 2,3-dioxygenase (IDO), as well as to pharmaceutical compositions comprising such compounds and to pharmaceutical methods of treatment comprising administration of such compounds and pharmaceutical compositions.

### CROSS REFERENCE TO RELATED APPLICATIONS

This application claims the benefit of U.S. Ser. No. 60/845,687, filed Sep. 19, 2006, and U.S. Ser. No. 60/900,827, filed Feb. 12, 2007, the disclosures of each of which are incorporated herein by reference in their entireties.

### TECHNICAL FIELD

This invention relates to modulators of indoleamine 2,3-dioxygenase (IDO), as well as to compositions and pharmaceutical methods thereof.

### BACKGROUND

Tryptophan (Trp) is an essential amino acid required for the biosynthesis of proteins, niacin and the neurotransmitter 5-hydroxytryptamine (serotonin). The enzyme indoleamine 2,3-dioxygenase (also known as INDO or IDO) catalyzes the first and rate limiting step in the degradation of L-tryptophan to N-formyl-kynurenine. In human cells, a depletion of Trp resulting from IDO activity is a prominent gamma interferon (IFN-γ)-inducible antimicrobial effector mechanism. IFN-γ stimulation induces activation of IDO, which leads to a depletion of Trp, thereby arresting the growth of Trp-dependent intracellular pathogens such as Toxoplasma gondii and Chlamydia trachomatis. IDO activity also has an antiproliferative effect on many tumor cells, and IDO induction has been observed in vivo during rejection of allogeneic tumors, indicating a possible role for this enzyme in the tumor rejection process (Daubener, et al., 1999, Adv. Exp. Med. Biol., 467: 517-24; Taylor, et al., 1991, FASEB J., 5: 2516-22).

It has been observed that HeLa cells co-cultured with peripheral blood lymphocytes (PBLs) acquire an immuno-inhibitory phenotype through up-regulation of IDO activity. A reduction in PBL proliferation upon treatment with interleukin-2 (IL2) was believed to result from IDO released by the tumor cells in response to IFNG secretion by the PBLs. This effect was reversed by treatment with 1-methyl-tryptophan (1MT), a specific IDO inhibitor. It was proposed that IDO activity in tumor cells may serve to impair antitumor responses (Logan, et al., 2002, Immunology, 105: 478-87).

Recently, an immunoregulatory role of Trp depletion has received much attention. Several lines of evidence suggest that IDO is involved in induction of immune tolerance. Studies of mammalian pregnancy, tumor resistance, chronic infections and autoimmune diseases have shown that cells expressing IDO can suppress T-cell responses and promote tolerance. Accelerated Trp catabolism has been observed in diseases and disorders associated with cellular immune activation, such as infection, malignancy, autoimmune diseases and AIDS, as well as during pregnancy. For example, increased levels of IFNs and elevated levels of urinary Trp metabolites have been observed in autoimmune diseases; it has been postulated that systemic or local depletion of Trp occurring in autoimmune diseases may relate to the degeneration and wasting symptoms of these diseases. In support of this hypothesis, high levels of IDO were observed in cells isolated from the synovia of arthritic joints. IFNs are also elevated in human immunodeficiency virus (HIV) patients and increasing IFN levels are associated with a worsening prognosis. Thus, it was proposed that IDO is induced chronically by HIV infection, and is further increased by opportunistic infections, and that the chronic loss of Trp initiates mechanisms responsible for cachexia, dementia and diarrhea and possibly immunosuppression of AIDS patients (Brown, et al., 1991, Adv. Exp. Med. Biol., 294: 425-35). To this end, it has recently been shown that IDO inhibition can enhance the levels of virus-specific T cells and, concomitantly, reduce the number of virally-infected macrophages in a mouse model of HIV (Portula et al., 2005, Blood, 106:2382-90).

IDO is believed to play a role in the immunosuppressive processes that prevent fetal rejection in utero. More than 40 years ago, it was observed that, during pregnancy, the genetically disparate mammalian conceptus survives in spite of what would be predicted by tissue transplantation immunology (Medawar, 1953, Symp. Soc. Exp. Biol. 7: 320-38). Anatomic separation of mother and fetus and antigenic immaturity of the fetus cannot fully explain fetal allograft survival. Recent attention has focused on immunologic tolerance of the mother. Because IDO is expressed by human syncytiotrophoblast cells and systemic tryptophan concentration falls during normal pregnancy, it was hypothesized that IDO expression at the maternal-fetal interface is necessary to prevent immunologic rejection of the fetal allografts. To test this hypothesis, pregnant mice (carrying syngeneic or allogeneic fetuses) were exposed to 1MT, and a rapid, T cell-induced rejection of all allogeneic concepti was observed. Thus, by catabolizing tryptophan, the mammalian conceptus appears to suppresses T-cell activity and defends itself against rejection, and blocking tryptophan catabolism during murine pregnancy allows maternal T cells to provoke fetal allograft rejection (Munn, et al., 1998, Science 281: 1191-3).

Further evidence for a tumoral immune resistance mechanism based on tryptophan degradation by IDO comes from the observation that most human tumors constitutively express IDO, and that expression of IDO by immunogenic mouse tumor cells prevents their rejection by preimmunized mice. This effect is accompanied by a lack of accumulation of specific T cells at the tumor site and can be partly reverted by systemic treatment of mice with an inhibitor of IDO, in the absence of noticeable toxicity. Thus, it was suggested that the efficacy of therapeutic vaccination of cancer patients might be improved by concomitant administration of an IDO inhibitor (Uyttenhove et al., 2003, Nature Med., 9: 1269-74). It has also been shown that the IDO inhibitor, 1-MT, can synergize with chemotherapeutic agents to reduce tumor growth in mice, suggesting that IDO inhibition may also enhance the anti-tumor activity of conventional cytotoxic therapies (Muller et al., 2005, Nature Med., 11:312-9).

One mechanism contributing to immunologic unresponsiveness toward tumors may be presentation of tumor antigens by tolerogenic host APCs. A subset of human IDO-expressing antigen-presenting cells (APCs) that coexpressed CD123 (IL3RA) and CCR6 and inhibited T-cell proliferation have also been described. Both mature and immature CD123-positive dendritic cells suppressed T-cell activity, and this IDO suppressive activity was blocked by 1MT (Munn, et al., 2002, Science 297: 1867-70). It has also been demonstrated that mouse tumor-draining lymph nodes (TDLNs) contain a subset of plasmacytoid dendritic cells (pDCs) that constitutively express immunosuppressive levels of IDO. Despite comprising only 0.5% of lymph node cells, in vitro, these pDCs potently suppressed T cell responses to antigens presented by the pDCs themselves and also, in a dominant fashion, suppressed T cell responses to third-party antigens presented by nonsuppressive APCs. Within the population of pDCs, the majority of the functional IDO-mediated suppressor activity segregated with a novel subset of pDCs coexpressing the B-lineage marker CD19. Thus, it was hypothesized that IDO-mediated suppression by pDCs in TDLNs creates a local microenvironment that is potently suppressive of host antitumor T cell responses (Munn, et al., 2004, J. Clin. Invest., 114(2): 280-90).

IDO degrades the indole moiety of tryptophan, serotonin and melatonin, and initiates the production of neuroactive and immunoregulatory metabolites, collectively known as kynurenines. By locally depleting tryptophan and increasing proapoptotic kynurenines, IDO expressed by dendritic cells (DCs) can greatly affect T-cell proliferation and survival. IDO induction in DCs could be a common mechanism of deletional tolerance driven by regulatory T cells. Because such tolerogenic responses can be expected to operate in a variety of physiopathological conditions, tryptophan metabolism and kynurenine production might represent a crucial interface between the immune and nervous systems (Grohmann, et al., 2003, Trends Immunol., 24: 242-8). In states of persistent immune activation, availability of free serum Trp is diminished and, as a consequence of reduced serotonin production, serotonergic functions may also be affected (Wirleitner, et al., 2003, Curr. Med. Chem., 10: 1581-91).

Interestingly, administration of interferon-α has been observed to induce neuropsychiatric side effects, such as depressive symptoms and changes in cognitive function. Direct influence on serotonergic neurotransmission may contribute to these side effects. In addition, because IDO activation leads to reduced levels of tryptophan, the precursor of serotonin (5-HT), IDO may play a role in these neuropsychiatric side effects by reducing central 5-HT synthesis. Furthermore, kynurenine metabolites such as 3-hydroxy-kynurenine (3-OH-KYN) and quinolinic acid (QUIN) have toxic effects on brain function. 3-OH-KYN is able to produce oxidative stress by increasing the production of reactive oxygen species (ROS), and QUIN may produce overstimulation of hippocampal N-methyl-D-aspartate (NMDA) receptors, which leads to apoptosis and hippocampal atrophy. Both ROS overproduction and hippocampal atrophy caused by NMDA overstimulation have been associated with depression (Wichers and Maes, 2004, J. Psychiatry Neurosci., 29: 11-17). Thus, IDO activity may play a role in depression.

Small molecule inhibitors of IDO are being developed to treat or prevent IDO-related diseases such as those described above. For example, PCT Publication WO 99/29310 reports methods for altering T cell-mediated immunity comprising altering local extracellular concentrations of tryptophan and tryptophan metabolites, using an inhibitor of IDO such as 1-methyl-DL-tryptophan, p-(3-benzofuranyl)-DL-alanine, p-[3-benzo(b)thienyl]-DL-alanine, and 6-nitro-L-tryptophan) (Munn, 1999). Reported in WO 03/087347, also published as European Patent 1501918, are methods of making antigen-presenting cells for enhancing or reducing T cell tolerance (Munn, 2003). Compounds having indoleamine-2,3-dioxygenase (IDO) inhibitory activity are further reported in WO 2004/094409; and U.S. Patent Application Publication No. 2004/0234623 is directed to methods of treating a subject with a cancer or an infection by the administration of an inhibitor of indoleamine-2,3-dioxygenase in combination with other therapeutic modalities.

In light of the experimental data indicating a role for IDO in immunosuppression, tumor resistance and/or rejection, chronic infections, HIV-infection, AIDS (including its manifestations such as cachexia, dementia and diarrhea), autoimmune diseases or disorders (such as rheumatoid arthritis), and immunologic tolerance and prevention of fetal rejection in utero, therapeutic agents aimed at suppression of tryptophan degradation by inhibiting IDO activity are desirable. Inhibitors of IDO can be used to activate T cells and therefore enhance T cell activation when the T cells are suppressed by pregnancy, malignancy or a virus such as HIV. Inhibition of IDO may also be an important treatment strategy for patients with neurological or neuropsychiatric diseases or disorders such as depression. The compounds, compositions and methods herein help meet the current need for IDO modulators.

### SUMMARY

The present invention provides, inter alia, compounds of Formula Ia:

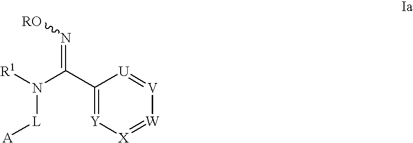

or pharmaceutically acceptable salts or prodrugs thereof, wherein constituent members are provided herein.

The present invention further provides compositions comprising a compound of Formula Ia and a pharmaceutically acceptable carrier.

The present invention further provides methods of modulating enzyme activity of IDO comprising contacting a compound of Formula Ia with the IDO.

The present invention further provides methods of treating IDO-associated diseases, including cancer, viral infection and depression, comprising administering to a patient a therapeutically effective amount of a compound of Formula Ia.

The present invention further provides methods of altering extracellular tryptophan levels in a mammal comprising administering to the mammal an effective amount of a compound of Formula Ia.

The present invention further provides methods of inhibiting immunosuppression, such as IDO-mediated immunosuppression, in a patient comprising administering to the patient an effective amount of a compound of Formula Ia.

The details of one or more embodiments of the invention are set forth in the accompanying drawings and the description below. Other features, objects, and advantages of the invention will be apparent from the description and drawings, and from the claims.

### DETAILED DESCRIPTION

The present invention provides compounds which are modulators of IDO having Formula Ia:

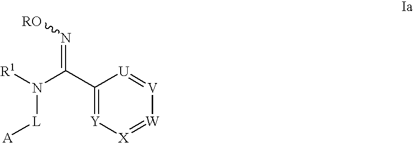

or pharmaceutically acceptable salts or prodrugs thereof, wherein:

U, V, W, X and Y are independently selected from N and CR^{x}, wherein 0, 1, 2 or 3 of U, V, W, X and Y are N, and the remainder of U, V, W, X and Y are CR^{x};

L is a bond, C₁₋₆ alkylene, C₂₋₆ alkenylene, C₂₋₆ alkynylene, (C₁₋₆ alkylene)ᵣ-O—(C₁₋₆ alkylene)ₛ, (C₁₋₆ alkylene)ᵣ-S—(C₁₋₆ alkylene)ₛ, (C₁₋₆ alkylene)ᵣ-NR^{j}—(C₁₋₆ alkylene)ₛ, (C₁₋₆ alkylene)ᵣ-CO—(C₁₋₆ alkylene)ₛ, (C₁₋₆ alkylene)ᵣ-COO—(C₁₋₆ alkylene)ₛ, (C₁₋₆ alkylene)ᵣ-CONR^{j}—(C₁₋₆ alkylene)ₛ, (C₁₋₆ alkylene)ᵣ-SO—(C₁₋₆ alkylene)ₛ, (C₁₋₆ alkylene)ᵣ-SO₂—(C₁₋₆ alkylene)ₛ, (C₁₋₆ alkylene)ᵣ-SONR^{j}—(C₁₋₆ alkylene)ₛ, (C₁₋₆ alkylene)ᵣ-SO₂NR^{j}—(C₁₋₆ alkylene)ₛ, or (C₁₋₆ alkylene)ᵣ-NR^{j}CONR^{k}—(C₁₋₆ alkylene)ₛ, wherein each of the C₁₋₆ alkylene, C₂₋₆ alkenylene, and C₂₋₆ alkynylene is optionally substituted by 1, 2, or 3 substituents independently selected from halo, CN, NO₂, N₃, SCN, OH, C₁₋₆ alkyl, C₁₋₆ haloalkyl, C₂₋₆ alkoxyalkyl, C₁₋₆ alkoxy, C₁₋₆ haloalkoxy, amino, C₁₋₆ alkylamino, and C₂₋₈ dialkylamino;

A is aryl, cycloalkyl, heteroaryl, or heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₁₋₆ hydroxyalkyl, C₁₋₆ cyanoalkyl, pentahalosulfanyl, Cy, CN, ORE, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, P(R^{f})₂, P(OR^{e})₂, P(O)R^{e}R^{f}, P(O)OR^{e}OR^{f}, S(O)R^{b}, S(O)NR^{c}R^{d}, S(O)₂R^{b}, NR^{c}S(O)₂R^{b}, and S(O)₂NR^{c}R^{d}, wherein said C₁₋₆ alkyl, C₂₋₆ alkenyl, and C₂₋₆ alkynyl is optionally substituted with 1, 2, or 3 substitutents independently selected from Cy, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{e}R^{f}, NR^{c}C(O)OR^{a}, C(═NRⁱ)NR^{c}R^{d}, NR^{c}C(═NRⁱ)NR^{c}R^{d}, P(R^{f})₂, P(OR^{e})₂, P(O)R^{e}R^{f}, P(O)OR^{e}OR^{f}, S(O)R^{b}, S(O)NR^{c}R^{d}, S(O)₂R^{b}, NR^{c}S(O)₂R^{b}, and S(O)₂NR^{c}R^{d};

R is H, C(O)R², C(O)OR³, or C(O)NR⁴R⁵;

R¹ is H or C₁₋₄ alkyl;

R² and R³ are independently selected from H, C₁₋₈ alkyl, aryl, heteroaryl, cycloalkyl, heterocycloalkyl, arylalkyl, heteroarylalkyl, cycloalkylalkyl, and heterocycloalkylalkyl, each optionally substituted by 1, 2, or 3 substituents independently selected from halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, and C₂₋₆ alkynyl;

R⁴ and R⁵ are independently selected from H, C₁₋₈ alkyl, aryl, heteroaryl, cycloalkyl, heterocycloalkyl, arylalkyl, heteroarylalkyl, cycloalkylalkyl, and heterocycloalkylalkyl, each optionally substituted by 1, 2, or 3 substituents independently selected from halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, and C₂₋₆ alkynyl; or

R⁴ and R⁵ together with the N atom to which they are attached form a 4-, 5-, 6- or 7-membered heterocycloalkyl group;

R^{x} is independently selected from H, halo, C₁₋₁₀ alkyl, C₂₋₆ alkenyl, C₂₋₆ alkynyl, pentahalosulfanyl, Cy¹, Cy¹—(C₁₋₆ alkyl), CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}NR^{c2}R^{d2}, NR^{c2}C(O)R^{b2}, NR^{c2}C(O)OR^{a2}, S(O)R^{b2}, S(O)NR^{c2}R^{d2}, S(O)₂R^{b2}, NR^{c2}S(O)₂R^{d2} and S(O)₂NR^{c2}R^{d2}; wherein said C₁₋₁₀ alkyl, C₂₋₆ alkenyl, or C₂₋₆ alkynyl is optionally substituted with 1, 2, 3, 4, or 5 substituents independently selected from halo, CN, NO₂, Cy¹, Cy¹—(C₁₋₆ alkyl)-, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{d2}, C(═NRⁱ)NR^{c2}R^{d2}, NR^{c2}C(═NRⁱ)NR^{c2}R^{d2}, P(R^{e2})₂, P(OR^{e2})₂, P(O)R^{e2}R^{f2}, P(O)OR^{e2}OR^{f2}, S(O)R^{b2}, S(O)NR^{c2}R^{d2}, S(O)₂R^{b2}, NR^{c2}S(O)₂R^{b2} and S(O)₂NR^{c2}R^{d2};

Cy and Cy¹ are independently selected from aryl, heteroaryl, cycloalkyl, and heterocycloalkyl, each optionally substituted by 1, 2, 3, 4 or 5 substituents independently selected from halo, C₁₋₄ alkyl, C₂₋₄ alkenyl, C₂₋₄ alkynyl, C₁₋₄ haloalkyl, pentahalosulfanyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, C(═NRⁱ)NR^{c1}R^{d1}, NR^{c1}C(═NRⁱ)NR^{c1}R^{d1}, P(R^{f1})₂, P(OR^{e1})₂, P(O)R^{e1}R^{f1}, P(O)OR^{e1}OR^{f1}, S(O)R^{b1}, S(O)NR^{c1}R^{d1}, S(O)₂R^{b1}, and S(O)₂NR^{c1}R^{d1};

R^{a}, R^{a1} and R^{a2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR)₂, P(O)R′R′, P(O)OR¹⁰R′, S(O)R′, S(O)NR′R′, S(O)₂R′, NR′S(O)₂R′, and S(O)₂NR′R′;

R^{b}, R^{b1} and R^{b2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR)₂, P(O)R′R′, P(O)OR¹⁰R′, S(O)R′, S(O)NR′R′, S(O)₂R′, NR′S(O)₂R′, and S(O)₂NR′R′;

R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′; or

R^{c} and R^{d} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;

R^{c1} and R^{d1} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′; or

R^{c1} and R^{d1} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;

R^{c2} and R^{d2} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;

or R^{c2} and R^{d2} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′,NR′C(O)R′,NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;

R^{e}, R^{e1} and R^{b2} are independently selected from H, C₁₋₆ alkyl, C₁₋₆ haloalkyl, C₂₋₆ alkenyl, (C₁₋₆ alkoxy)-C₁₋₆ alkyl, C₂₋₆ alkynyl, aryl, cycloalkyl, heteroaryl, heterocycloalkyl, arylalkyl, cycloalkylalkyl, heteroarylalkyl, and heterocycloalkylalkyl;

R^{f}, R^{f1} and R^{f2} are independently selected from H, C₁₋₆ alkyl, C₁₋₆ haloalkyl, C₂₋₆ alkenyl, C₂₋₆ alkynyl, aryl, cycloalkyl, heteroaryl, and heterocycloalkyl;

Rⁱ is H, CN, or NO₂;

R^{j} and R^{k} are independently selected from H and C₁₋₆ alkyl;

R′ is H, C₁₋₆ alkyl, C₁₋₆ haloalkyl, C₂₋₆ alkenyl, C₂₋₆ alkynyl, aryl, cycloalkyl, heteroaryl, and heterocycloalkyl;

r is 0 or 1; and

s is 0 or 1.

The present invention provides compounds which are modulators of IDO having Formula I:

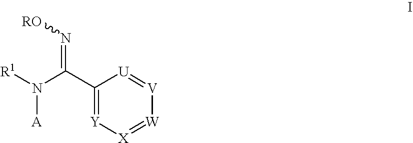

or pharmaceutically acceptable salts or prodrugs thereof, wherein:

U, V, W, X and Y are independently selected from N and CR^{x}, wherein 0, 1, 2 or 3 of U, V, W, X and Y are N, and the remainder of U, V, W, X and Y are CR^{x};

A is aryl, cycloalkyl, heteroaryl, or heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₁₋₆ hydroxyalkyl, C₁₋₆ cyanoalkyl, pentahalosulfanyl, Cy, CN, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, P(R^{f})₂, P(OR^{e})₂, P(O)R^{e}R^{f}, P(O)OR^{e}OR^{f}, S(O)R^{b}, S(O)NR^{c}R^{d}, S(O)₂R^{b}, NR^{c}S(O)₂R^{b}, and S(O)₂NR^{c}R^{d}, wherein said C₁₋₆ alkyl, C₂₋₆ alkenyl, and C₂₋₆ alkynyl is optionally substituted with 1, 2, or 3 substitutents independently selected from Cy, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, P(R^{f})₂, P(OR^{e})₂, P(O)R^{e}R^{f}, P(O)OR^{e}OR^{f}, S(O)R^{b}, S(O)NR^{c}R^{d}, S(O)₂R^{b}, NR^{c}S(O)₂R^{b}, and S(O)₂NR^{c}R^{d};

R¹ is H, C(O)R², C(O)OR³, or C(O)NR⁴R⁵;

R¹ is H or C₁₋₄ alkyl;

R² and R³ are independently selected from H, C₁₋₈ alkyl, aryl, heteroaryl, cycloalkyl, heterocycloalkyl, arylalkyl, heteroarylalkyl, cycloalkylalkyl, and heterocycloalkylalkyl, each optionally substituted by 1, 2, or 3 substituents independently selected from halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, and C₂₋₆ alkynyl;

R⁴ and R⁵ are independently selected from H, C₁₋₈ alkyl, aryl, heteroaryl, cycloalkyl, heterocycloalkyl, arylalkyl, heteroarylalkyl, cycloalkylalkyl, and heterocycloalkylalkyl, each optionally substituted by 1, 2, or 3 substituents independently selected from halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, and C₂₋₆ alkynyl; or

R⁴ and R⁵ together with the N atom to which they are attached form a 4-, 5-, 6- or 7-membered heterocycloalkyl group;

Cy and Cy¹ are independently selected from aryl, heteroaryl, cycloalkyl, and heterocycloalkyl, each optionally substituted by 1, 2, 3, 4 or 5 substituents independently selected from halo, C₁₋₄ alkyl, C₂₋₄ alkenyl, C₂₋₄ alkynyl, C₁₋₄ haloalkyl, pentahalosulfanyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, C(═NRⁱ)NR^{c1}R^{d1}NR^{c1}C(═NRⁱ)NR^{c1}R^{d1}, P(R^{f1})₂, P(OR^{e1})₂, P(O)R^{e1}R^{f1}, P(O)OR^{e1}OR^{f1}, S(O)R^{b1}, S(O)NR^{c1}R^{d2}, S(O)₂R^{e1}, and S(O)₂NR^{e1}R^{d1};

R^{a}, R^{a1} and R^{a2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, C₁₋₆ alkoxy, amino, halo, C₁₋₆ alkyl, aryl, arylalkyl, heteroaryl, heteroarylalkyl, cycloalkyl, and heterocycloalkyl;

R^{b}, R^{b1} and R^{b2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, amino, halo, C₁₋₆ alkyl, C₁₋₆ haloalkyl, aryl, arylalkyl, heteroaryl, heteroarylalkyl, cycloalkyl, and heterocycloalkyl;

R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl, or heterocycloalkyl; or

R^{c} and R^{d} together with the N atom to which they are attached form a 4-, 5-, 6- or 7-membered heterocycloalkyl group;

R^{c1} and R^{d1} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl, or heterocycloalkyl; or

R^{c1} and R^{d1} together with the N atom to which they are attached form a 4-, 5-, 6- or 7-membered heterocycloalkyl group;

R^{c2} and R^{d2} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with OH, amino, halo, C₁₋₆ alkyl, C₁₋₆ alkoxy, C₁₋₆ haloalkyl, aryl, arylalkyl, heteroaryl, heteroarylalkyl, cycloalkyl, or heterocycloalkyl;

or R^{c2} and R^{d2} together with the N atom to which they are attached form a 4-, 5-, 6- or 7-membered heterocycloalkyl group;

R^{e}, R^{e1} and R^{b2} are independently selected from H, C₁₋₆ alkyl, C₁₋₆ haloalkyl, C₂₋₆ alkenyl, (C₁₋₆ alkoxy)-C₁₋₆ alkyl, C₂₋₆ alkynyl, aryl, cycloalkyl, heteroaryl, heterocycloalkyl, arylalkyl, cycloalkylalkyl, heteroarylalkyl, and heterocycloalkylalkyl;

R^{f}, R^{f1} and R^{f2} are independently selected from H, C₁₋₆ alkyl, C₁₋₆ haloalkyl, C₂₋₆ alkenyl, C₂₋₆ alkynyl, aryl, cycloalkyl, heteroaryl, and heterocycloalkyl;

Rⁱ is H, CN, or NO₂; and

R^{x} is independently selected from H, halo, C₁₋₁₀ alkyl, C₂₋₆ alkenyl, C₂₋₆ alkynyl, pentahalosulfanyl, Cy¹, Cy¹—(C₁₋₆ alkyl), CN, NO₂, OR^{a2}, SR², C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, S(O)R^{b2}, S(O)NR^{c2}R^{d2}, S(O)₂R^{b2}, and S(O)₂NR^{c2}R^{d2}; wherein said C₁₋₁₀ alkyl, C₂₋₆ alkenyl, or C₂₋₆ alkynyl is optionally substituted with 1, 2, 3, 4, or 5 substituents independently selected from halo, CN, NO₂, Cy¹, Cy¹—(C₁₋₆ alkyl)-, OR^{a2}, SR^{a2}, C(O)R^{b2}, C(O)NR^{c2}R^{d2}, C(O)OR^{a2}, OC(O)R^{b2}OC(O)NR^{c2}R^{d2}, NR^{c2}R^{d2}, NR^{c2}C(O)R^{b2}NR^{c2}C(O)OR^{a2}, C(═NRⁱ)NR^{c2}R^{d2}, NR^{c2}C(═NRⁱ)NR^{c2}R^{d2}, P(R^{f2})₂, P(OR^{e2})₂, P(O)R^{e2}R^{f2}P(O)OR^{e2}OR^{f2}, S(O)NR^{c2}R^{d2}, S(O)₂R^{b2}, and S(O)₂NR^{c2}R^{d2}.

In some embodiments, when the ring containing U, V, W, X and Y is pyrimidyl, unsubstituted aryl, or unsubstituted pyridyl, then A is other than unsubstituted phenyl.

In some embodiments, when the ring containing U, V, W, X and Y is pyrimidyl, unsubstituted aryl, or unsubstituted pyridyl, and L is a bond, then A is other than unsubstituted phenyl.

In some embodiments, when A is a phenyl group substituted by CH₃, then the ring containing U, V, W, X and Y is other than perfluorophenyl.

In some embodiments, when A is a phenyl group substituted by CH₃, and L is a bond, then the ring containing U, V, W, X and Y is other than perfluorophenyl.

In some embodiments, when the ring containing U, V, W, X and Y is unsubstituted phenyl, or phenyl substituted at the 3-position by CN, NO₂, CF₃, OCF₂CHF₂ or Cl, then A is other than phenyl substituted at the 3-position by Cl or F or at the 4-position by SO₂NR^{c}R^{d}.

In some embodiments, when the ring containing U, V, W, X and Y is unsubstituted phenyl, or phenyl substituted at the 3-position by CN, NO₂, CF₃, OCF₂CHF₂ or Cl, and L is a bond, then A is other than phenyl substituted at the 3-position by Cl or F or at the 4-position by SO₂NR^{c}R^{d}.

In some embodiments, when the ring containing U, V, W, X and Y is unsubstituted phenyl, or phenyl substituted at the 3-position by CN, NO₂, CF₃, OCF₂CHF₂ or Cl, then A is other than phenyl substituted by Cl, F or SO₂NR^{c}R^{d}.

In some embodiments, when the ring containing U, V, W, X and Y is unsubstituted phenyl, or phenyl substituted at the 3-position by CN, NO₂, CF₃, OCF₂CHF₂ or Cl, and L is a bond, then A is other than phenyl substituted by Cl, F or SO₂NR^{c}R^{d}.

In some embodiments, when the ring containing U, V, W, X and Y is unsubstituted phenyl, or phenyl substituted by CN, NO₂, CF₃, OCF₂CHF₂ or Cl, then A is other than phenyl substituted by Cl, F or SO₂NR^{c}R^{d}.

In some embodiments, when the ring containing U, V, W, X and Y is unsubstituted phenyl, or phenyl substituted by CN, NO₂, CF₃, OCF₂CHF₂ or Cl, and L is a bond, then A is other than phenyl substituted by Cl, F or SO₂NR^{c}R^{d}.

In some embodiments, when the ring containing U, V, W, X and Y is unsubstituted pyridyl, then A is other than unsubstituted naphthyl or phenyl substituted by F, CF₃ or CH₃

In some embodiments, when the ring containing U, V, W, X and Y is unsubstituted pyridyl, and L is a bond, then A is other than unsubstituted naphthyl or phenyl substituted by F, CF₃ or CH₃.

In some embodiments, when the ring containing U, V, W, X and Y is pyridyl substituted with a CF₃ group, then A is other than phenyl substituted at the 2-position by Cl or CH₃, or phenyl substituted at the 3-position by C₁, CF₃ or O-heteroaryl.

In some embodiments, when the ring containing U, V, W, X and Y is pyridyl substituted with a CF₃ group, and L is a bond, then A is other than phenyl substituted at the 2-position by C₁ or CH₃, or phenyl substituted at the 3-position by C₁, CF₃ or O-heteroaryl.

In some embodiments, when the ring containing U, V, W, X and Y is pyridyl substituted with a CF₃ group, then A is other than phenyl substituted at the 2-position by C₁ or CH₃, or phenyl substituted at the 3-position by C₁, CF₃ or O-heteroaryl.

In some embodiments, when the ring containing U, V, W, X and Y is pyridyl substituted with a CF₃ group, and L is a bond, then A is other than phenyl substituted at the 2-position by C₁ or CH₃, or phenyl substituted at the 3-position by C₁, CF₃ or O-heteroaryl.

In some embodiments, when the ring containing U, V, W, X and Y is pyridyl substituted with a CF₃ group, then A is other than phenyl substituted by C₁ or CH₃, or phenyl substituted at the 3-position by C₁, CF₃ or O-heteroaryl.

In some embodiments, when the ring containing U, V, W, X and Y is pyridyl substituted with a CF₃ group, and L is a bond, then A is other than phenyl substituted by C₁ or CH₃, or phenyl substituted at the 3-position by C₁, CF₃ or O-heteroaryl.

In some embodiments, when the ring containing U, V, W, X and Y is pyridyl substituted with a CF₃ group, then A is other than phenyl substituted by Cl, CH₃, CF₃ or O-heteroaryl.

In some embodiments, when the ring containing U, V, W, X and Y is pyridyl substituted with a CF₃ group, and L is a bond, then A is other than phenyl substituted by Cl, CH₃, CF₃ or O-heteroaryl.

In some embodiments, when the ring containing U, V, W, X and Y is phenyl substituted with at least one F, Cl, Br, CF₃, CN, NO₂, S(O)₂R^{b2}, and S(O)₂NR^{c2}R^{d2}, C(O)OR^{a2}, Cy¹, OR^{a2}, SR^{a2}, C₁₋₁₀ alkyl, C₂₋₆ alkenyl, or C₂₋₆ alkynyl, and L is a bond; then A is optionally substituted cycloalkyl, optionally substituted heteroaryl, optionally substituted heterocycloalkyl, aryl substituted by at least one CF₃, or aryl substituted by at least two substituents.

In some embodiments, U, V, W, X and Y are CR^{x}.

In some embodiments, U is N.

In some embodiments, U is N and X is N.

In some embodiments, U is N and W is N.

In some embodiments, U is N, W is N and Y is N.

In some embodiments, V is N.

In some embodiments, V is N and X is N.

In some embodiments, W is N.

In some embodiments, U and V are both N.

In some embodiments, L is a bond, C₁₋₆ alkylene, (C₁₋₆ alkylene)ᵣ-CO—(C₁₋₆ alkylene)ₛ, C₁₋₆ alkylene)ᵣ-NR^{j}—(C₁₋₆ alkylene)ₛ, or (C₁₋₆ alkylene)ᵣ-SO₂—(C₁₋₆ alkylene)ₛ.

In some embodiments, L is a bond or C₁₋₆ alkylene.

In some embodiments, L is a bond.

In some embodiments, L is C₁₋₆ alkylene.

In some embodiments, L is methylene.

In some embodiments, L is (C₁₋₆ alkylene)ᵣ-O—(C₁₋₆ alkylene)ₛ.

In some embodiments, L is (C₁₋₆ alkylene)ᵣ-S—(C₁₋₆ alkylene)ₛ.

In some embodiments, L is C₁₋₆ alkylene)ᵣ-NR^{j}—(C₁₋₆ alkylene)ₛ.

In some embodiments, L is (C₁₋₆ alkylene)ᵣ-CO—(C₁₋₆ alkylene)ₛ.

In some embodiments, L is (C₁₋₆ alkylene)ᵣ-COO—(C₁₋₆ alkylene)ₛ.

In some embodiments, L is (C₁₋₆ alkylene)ᵣ-CONR^{j}—(C₁₋₆ alkylene)ₛ.

In some embodiments, L is (C₁₋₆ alkylene)ᵣ-SO—(C₁₋₆ alkylene)ₛ.

In some embodiments, L is (C₁₋₆ alkylene)ᵣ-SO₂—(C₁₋₆ alkylene)ₛ.

In some embodiments, L is C₁₋₆ alkylene)ᵣ-SONR^{j}—(C₁₋₆ alkylene)ₛ.

In some embodiments, L is C₁₋₆ alkylene)ᵣ-SO₂NR^{j}—(C₁₋₆ alkylene)ₛ.

In some embodiments, L is (C₁₋₆ alkylene)ᵣ-NR^{j}CONR^{k}—(C₁₋₆ alkylene)ₛ.

In some embodiments, r is 0.

In some embodiments, r is 1

In some embodiments, s is 0.

In some embodiments, s is 1.

In some embodiments, A is aryl or heteroaryl, optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₁₋₆ hydroxyalkyl, C₁₋₆ cyanoalkyl, Cy, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, P(R^{f})₂, P(OR^{e})₂, P(O)R^{e}R^{f}, P(O)OR^{e}OR^{f}, S(O)R^{b}, S(O)NR^{c}R^{d}, S(O)₂R^{b}, NR^{c}S(O)₂R^{b}, and S(O)₂NR^{c}R^{d}, wherein said C₁₋₆ alkyl, C₂₋₆ alkenyl, and C₂₋₆ alkynyl is optionally substituted with 1, 2, or 3 substituents independently selected from Cy, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{d}, C(═NRⁱ)NR^{c}R^{d}, NR^{c}C(═NRⁱ)NR^{c}R^{d}, P(R^{f})₂, P(OR^{e})₂, P(O)R^{e}R^{f}, P(O)OR^{e}OR^{f}, S(O)R^{b}, S(O)NR^{c}R^{d}, S(O)₂R^{b}, NR^{c}S(O)₂R^{b}, and S(O)₂NR^{c}R^{d}.

In some embodiments, A is phenyl optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₁₋₆ hydroxyalkyl, C₁₋₆ cyanoalkyl, Cy, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}NR^{c}C(═NRⁱ)NR^{c}R^{d}, P(R^{f})₂, P(OR^{e})₂, P(O)R^{e}R^{f}, P(O)OR^{e}OR^{f}, S(O)R^{b}, S(O)NR^{c}R^{d}, S(O)₂R^{b}, NR^{c}S(O)₂R^{e}, and S(O)₂NR^{c}R^{d}, wherein said C₁₋₆ alkyl, C₂₋₆ alkenyl, and C₂₋₆ alkynyl is optionally substituted with 1, 2, or 3 substituents independently selected from Cy, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, P(R^{f})₂, P(OR^{e})₂, P(O)R^{e}R^{f}, P(O)OR^{e}OR^{f}, S(O)R^{b}, S(O)NR^{c}R^{d}, S(O)₂R^{b}, NR^{c}S(O)₂R^{b}, and S(O)₂NR^{c}R^{d}.

In some embodiments, A is phenyl optionally substituted by 1, 2, 3, 4, or 5 substituents independently selected from halo, C₁₋₆ alkyl, C₁₋₆ haloalkyl, C₁₋₆ hydroxyalkyl, C₁₋₆ cyanoalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{d}, S(O)R^{b}, S(O)NR^{c}R^{d}, S(O)₂R^{b}, NR^{c}S(O)₂R^{b}, and S(O)₂NR^{c}R^{d}.

In some embodiments, A is phenyl optionally substituted by 1, 2, 3, 4, or 5 substituents independently selected from halo, C₁₋₆ alkyl and C₁₋₆ haloalkyl.

In some embodiments, A is optionally substituted cycloalkyl, optionally substituted heteroaryl, optionally substituted heterocycloalkyl, aryl substituted by at least one CF₃, or aryl substituted by at least two substituents, wherein the substituents are independently selected from those listed for A above.

In some embodiments, A is aryl substituted by at least two substituents, wherein the substituents are independently selected from those listed for A above.

In some embodiments, A is phenyl substituted by at least two substituents, wherein the substituents are independently selected from those listed for A above.

In some embodiments, A is aryl substituted by at least two halo or by at least one CF₃

In some embodiments, A is phenyl substituted by at least two halo or by at least one CF₃.

In some embodiments, A is aryl substituted by at least two halo.

In some embodiments, A is phenyl substituted by at least two halo.

In some embodiments, A is aryl substituted by at least one CF₃.

In some embodiments, A is phenyl substituted by at least one CF₃.

In some embodiments, Cy is aryl, heteroaryl, cycloalkyl, or heterocycloalkyl, each optionally substituted by 1, 2, 3, 4 or 5 substituents independently selected from halo, C₁₋₄ alkyl, C₂₋₄ alkenyl, C₂₋₄ alkynyl, C₁₋₄ haloalkyl, CN, NO₂, OR^{a1}, SR^{a}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, S(O)R^{b1}, S(O)NR^{c1}R^{d1}, S(O)₂R^{b1}, and S(O)₂NR^{c1}R^{d1}.

In some embodiments, Cy¹ is aryl, heteroaryl, cycloalkyl, or heterocycloalkyl, each optionally substituted by 1, 2, 3, 4 or 5 substituents independently selected from halo, C₁₋₄ alkyl, C₂₋₄ alkenyl, C₂₋₄ alkynyl, C₁₋₄ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d2}, NR^{c1}R^{d2}, NR^{c1}C(O)R^{b1}, NR^{c1}C(O)OR^{a1}, S(O)R^{b1}, S(O)NR^{c1}R^{d1}, S(O)₂R^{b1}, and S(O)₂NR^{c1}R^{d1}.

In some embodiments, R¹ is H.

In some embodiments, R is H.

In some embodiments, R^{x} is independently selected from H, halo, C₁₋₁₀ alkyl, C₂₋₆ alkenyl, C₂₋₆ alkynyl, pentahalosulfanyl, Cy¹, Cy¹-(C₁₋₆ alkyl), CN, NO₂, OR^{a2}, SR^{a2}, C(O)R^{b2}, C(O)NR^{c2}R^{d2}, C(O)OR^{a2}, OC(O)R^{b2}OC(O)NR^{c2}R^{d2}, NR^{c2}R^{d2}, NR^{c2}C(O)R^{b2} NR^{c2}C(O)OR^{a2}, S(O)R^{b2}, S(O)NR^{c2}R^{d2}, S(O)₂R^{b2}, and S(O)₂NR^{c2}R^{d2}; wherein said C₁₋₁₀ alkyl, C₂₋₆ alkenyl, or C₂₋₆ alkynyl is optionally substituted with 1, 2, 3, 4, or 5 substituents independently selected from halo, CN, NO₂, Cy¹, Cy¹-(C₁₋₆ alkyl)-, OR^{a2}, SR^{a2}, C(O)R^{b2}, C(O)NR^{c2}R^{d2}, C(O)OR^{a2}, OC(O)R^{b2}, OC(O)NR^{c2}R^{d2}NR^{c2}R^{d2}NR^{c2}C(O)R^{b2} NR^{c2}C(O)OR^{a2}, C(═NRⁱ)NR^{c2}R^{d2}, NR^{c2}C(═NRⁱ)NR^{c2}R^{d2}, P(R^{f2})₂, P(OR^{e2})₂, P(O)R^{e2}R^{f2}, P(O)OR^{e2}OR^{f2}, S(O)R^{b2}, S(O)NR^{c2}R^{d2}, S(O)₂R^{b2}, and S(O)₂NR^{c2}R^{d2};

In some embodiments, R^{x} is independently selected from H, halo, C₁₋₁₀ alkyl, C₂₋₆ alkenyl, C₂₋₆ alkynyl, pentahalosulfanyl, Cy¹, Cy¹-(C₁₋₆ alkyl), CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, N^{c2}C(O)R^{b2}, NR^{c2}C(O)OR^{a2}, S(O)R^{b2}, S(O)NR^{c2}R^{d2}, S(O)₂R^{b2}, and S(O)₂NR^{c2}R^{d2}; wherein said C₁₋₁₀ alkyl, C₂₋₆ alkenyl, or C₂₋₆ alkynyl is optionally substituted with 1, 2, 3, 4, or 5 substituents independently selected from CN, NO₂, Cy¹, Cy¹-(C₁₋₆ alkyl)-, OR^{a2}, SR², C(O)R^{b2}, C(O)NR^{c2}R^{d2}, C(O)OR^{a2}, OC(O)R^{b2}, OC(O)NR^{c2}R^{d2}NR^{c2}R^{d2}, NR^{c2}C(O)R^{b2}, NR^{c2}C(O)OR^{a2}, C(═NRⁱ)NR^{c2}R^{d2}, NR^{c2}C(═NRⁱ)NR^{c2}R^{d2}, P(R²)₂, p(OR^{b2})₂, P(O)R^{e2}R^{f2}, P(O)OR^{e2}OR^{f2}, S(O)R^{b2}, S(O)NR^{c2}R^{d2}, S(O)₂R^{b2}, and S(O)₂NR^{c2}R^{d2}.

In some embodiments, R^{x} is independently selected from H, pentahalosulfanyl, C(O)R^{b2}, C(O)NR^{c2}R^{d2}, OC(O)R^{b2}, OC(O)NR^{c2}R^{d2}NR^{c2}C(O)R^{b2}, NR^{c2}C(O)OR^{a2}, S(O)R^{b2}, and S(O)NR^{c2}R^{d2}; wherein said C₁₋₁₀ alkyl, C₂₋₆ alkenyl, or C₂₋₆ alkynyl is optionally substituted with 1, 2, 3, 4, or 5 substituents independently selected from CN, NO₂, Cy¹, Cy¹-(C₁₋₆ alkyl)-, OR^{a2}, SR^{b2}, C(O)R^{b2}, C(O)NR^{c2}R^{d2}, C(O)OR^{b2}, OC(O)R^{b2}, OC(O)NR^{c2}R^{d2}, NR^{c2}R^{d2}, NR^{c2}C(O)R^{b2}, NR^{c2}C(O)OR^{b2}, C(═NRⁱ)NR^{c2}R^{d2}, NR^{c2}C(═NRⁱ)NR^{c2}R^{d2}, P(R^{f2})₂, P(OR^{e2})₂, P(O)R^{e2}R^{f2}, P(O)OR^{e2}OR^{f2}, S(O)R^{b2}, S(O)NR^{c2}R^{d2}, S(O)₂R^{b2}, and S(O)₂NR^{c2}R^{d2}.

In some embodiments, R^{x} is independently selected from H, CN, NO₂, OR^{a2}, NR^{c2}R^{d2}.

In some embodiments, R^{x} is independently selected from H, OR^{a2} and NR^{c2}R^{d2}, and R^{a2}, R^{c2} and R^{d2} are independently selected from H, C₁₋₆ alkyl, arylalkyl and heteroarylalkyl.

In some embodiments, R^{x} is independently selected from H, O-aryl, O-arylalkyl, O-heteroarylalkyl, NH-arylalkyl and NH-heteroarylalkyl, wherein said aryl, arylalkyl, heteroaryl, and heteroarylalkyl are optionally substituted with 1, 2, or 3 substituents independently selected from halo and C₁₋₆ alkoxy.

In some embodiments, R^{x} is independently selected from H, phenoxy, methoxy, benzyloxy, amino, benzylamino and 4-methoxybenzylamino.

In some embodiments, R^{c2} and R^{d2} are independently selected from H, aryl, heteroaryl, arylalkyl, and heteroarylalkyl, wherein said aryl, heteroaryl, arylalkyl, heteroarylalkyl is optionally substituted with 1, 2, or 3 substituents independently selected from OH, amino, halo, C₁₋₆ alkyl, or C₁₋₆ alkoxy.

In some embodiments, R^{a2} is selected from H, C₁₋₆ alkyl, C₁₋₆ haloalkyl, aryl, heteroaryl, arylalkyl, or heteroarylalkyl, wherein said C₁₋₆ alkyl, C₁₋₆ haloalkyl, aryl, heteroaryl, arylalkyl, and heteroarylalkyl is optionally substituted with 1, 2, or 3 substituents independently selected from OH, C₁₋₆ alkoxy, amino, halo, C₁₋₆ alkyl, aryl, arylalkyl, heteroaryl, heteroarylalkyl, cycloalkyl and heterocycloalkyl.

In some embodiments, R^{a2} is selected from aryl, heteroaryl, arylalkyl, or heteroarylalkyl, wherein said aryl, heteroaryl, arylalkyl, and heteroarylalkyl is optionally substituted with 1, 2, or 3 substituents independently selected from amino, halo and C₁₋₆ alkoxy.

In some embodiments, R^{a2} is selected from phenyl or phenylalkyl, wherein said phenyl and phenylalkyl is optionally substituted with 1, 2, or 3 substituents independently selected from halo and C₁₋₆ alkoxy.

In some embodiments, R^{a}, R^{a1} and R^{a2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, C₁₋₆ alkoxy, amino, halo, C₁₋₆ alkyl, aryl, arylalkyl, heteroaryl, heteroarylalkyl, cycloalkyl, and heterocycloalkyl;

R^{b}, R^{b1} and R^{b2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from OH, amino, halo, C₁₋₆ alkyl, C₁₋₆ haloalkyl, aryl, arylalkyl, heteroaryl, heteroarylalkyl, cycloalkyl, and heterocycloalkyl;

R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl, or heterocycloalkyl; or

R^{c} and R^{d} together with the N atom to which they are attached form a 4-, 5-, 6- or 7-membered heterocycloalkyl group;

R^{c1} and R^{d1} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with OH, amino, halo, C₁₋₆ alkyl, C₁₋₆ haloalkyl, aryl, arylalkyl, heteroaryl, heteroarylalkyl, cycloalkyl, or heterocycloalkyl; or

R^{c1} and R^{d1} together with the N atom to which they are attached form a 4-, 5-, 6- or 7-membered heterocycloalkyl group; and

R^{c2} and R^{d2} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with OH, amino, halo, C₁₋₆ alkyl, C₁₋₆ alkoxy, C₁₋₆ haloalkyl, aryl, arylalkyl, heteroaryl, heteroarylalkyl, cycloalkyl, or heterocycloalkyl;

or R^{c2} and R^{d2} together with the N atom to which they are attached form a 4-, 5-, 6- or 7-membered heterocycloalkyl group.

In some embodiments, the compounds of the invention have Formula IIIa:

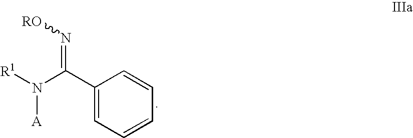

In some embodiments, the compounds of the invention have Formula IIIb:

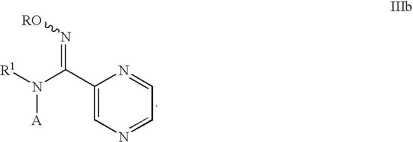

In some embodiments, the compounds of the invention have Formula IIIc:

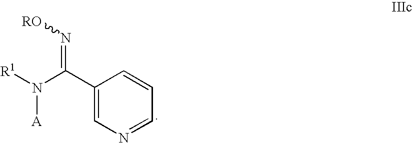

In some embodiments, the compounds of the invention have Formula IIId:

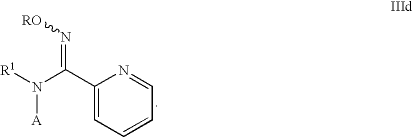

In some embodiments, the compounds of the invention have Formula IIIe:

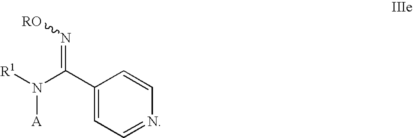

In some embodiments, the compounds of the invention have Formula IVa:

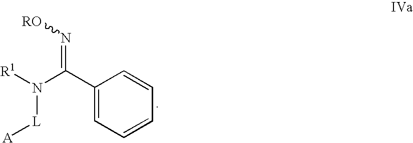

In some embodiments, the compounds of the invention have Formula IVb:

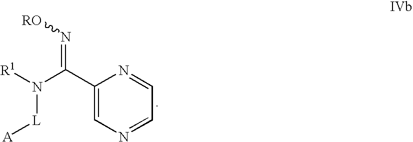

In some embodiments, the compounds of the invention have Formula IVc:

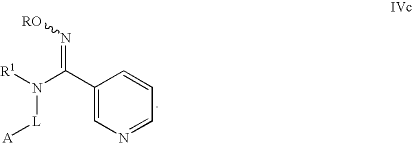

In some embodiments, the compounds of the invention have Formula IVd:

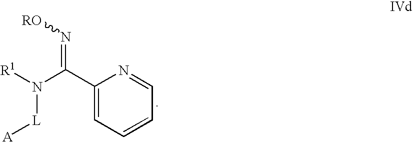

In some embodiments, the compounds of the invention have Formula IVe:

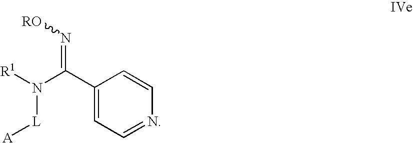

In some embodiments, the compounds of the invention have Formula IIIf:

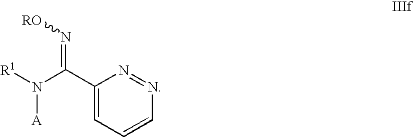

In some embodiments, the compounds of the invention have Formula IVf:

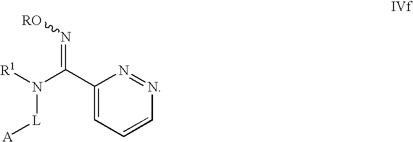

In another embodiment of the invention, there is provided a pharmaceutical composition comprising a compound of Formula Ia, or pharmaceutically acceptable salt thereof, and at least one pharmaceutically acceptable carrier.

In another embodiment of the invention, there is provided a pharmaceutical composition comprising a compound of Formula IIIa, IIIb, IIIc, IIId, IIIe, or IIIf, or pharmaceutically acceptable salt thereof, and at least one pharmaceutically acceptable carrier.

In another embodiment of the invention, there is provided a pharmaceutical composition comprising a compound of Formula IVa, IVb, IVc, IVd, IVe, or IVf, or pharmaceutically acceptable salt thereof, and at least one pharmaceutically acceptable carrier.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., npentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms. A linking alkyl group is referred to herein as “alkylene.”

As used herein, “alkenyl” refers to an alkyl group having one or more double carbon-carbon bonds. Example alkenyl groups include ethenyl, propenyl, and the like. A linking alkenyl group is referred to herein as “alkenylene.”

As used herein, “alkynyl” refers to an alkyl group having one or more triple carbon-carbon bonds. Example alkynyl groups include ethynyl, propynyl, and the like.

As used herein, “haloalkyl” refers to an alkyl group having one or more halogen substituents, wherein the halogens may be the same or different. Example haloalkyl groups include CF₃, C₂F₅, CHF₂, CCl₃, CHCl₂, CFCl₂, C₂Cl₅, and the like. A linking alkynyl group is referred to herein as “alkynylene.”

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocycles. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of cyclopentane, cyclopentene, cyclohexane, and the like. A cycloalkyl group having one or more fused aromatic rings can be attached though either the aromatic or non-aromatic portion. One or more ring-forming carbon atoms of a cycloalkyl group can be oxidized, for example, having an oxo or sulfido substituent. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like.

As used herein, a “heteroaryl” group refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heterocycloalkyl” refers to a non-aromatic heterocycle where one or more of the ring-forming atoms is a heteroatom such as an O, N, or S atom. Heterocycloalkyl groups can include mono- or polycyclic (e.g., having 2, 3 or 4 fused rings) ring systems as well as spirocycles. Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles. A heterocycloalkyl group having one or more fused aromatic rings can be attached though either the aromatic or non-aromatic portion. In some embodiments, the heterocycloalkyl group has from 1 to about 20 carbon atoms, and in further embodiments from about 3 to about 20 carbon atoms. In some embodiments, the heterocycloalkyl group contains 3 to about 20, 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “halo” or “halogen” includes fluoro, chloro, bromo, and iodo.

As used herein, “hydroxyalkyl” refers to an alkyl group substituted with a hydroxyl group.

As used herein, “cyanoalkyl” refers to an alkyl group substituted with a cyano group.

As used herein, “alkoxy” refers to an —O-alkyl group. Example alkoxy groups include methoxy, ethoxy, propoxy (e.g., n-propoxy and isopropoxy), t-butoxy, and the like.

As used herein, “arylalkyl” refers to alkyl substituted by aryl and “cycloalkylalkyl” refers to alkyl substituted by cycloalkyl. An example arylalkyl group is benzyl.

As used herein, “heteroarylalkyl” refers to alkyl substituted by heteroaryl and “heterocycloalkylalkyl” refers to alkyl substituted by heterocycloalkyl.

As used herein, “pentahalosulfanyl” refers to moieties of formula —SX₅ where each X is independently selected from F, Cl, Br, or I. For methods of preparing compounds containing pentahalosulfanyl groups see, e.g. Org. Lett. 2002, 4, 3013. An example pentahalosulfanyl is SF₅.

As used herein, “amino” refers to NH₂.

As used herein, “alkylamino” refers to an amino group substituted by an alkyl group.

As used herein, “dialkylamino” refers to an amino group substituted by two alkyl groups.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium.

The term “compound” is meant to include all stereoisomers, geometric isomers, isotopes, tautomers, and resonance structures of the chemical formula depicted unless otherwise indicated.

Compounds of the invention can be present together with other substances, such as with water or solvent in the form of hydrates or solvates, or present in isolated form.

In some embodiments, the compounds of the invention, and salts thereof, are substantially isolated. By “substantially isolated” is meant that the compound is at least partially or substantially separated from the environment in which it was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington s Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes prodrugs of the compounds described herein. As used herein, “prodrugs” refer to any covalently bonded carriers which release the active parent drug when administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

### Synthesis

The novel compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance (NMR) spectroscopy (e.g., ¹H or ¹³C) infrared spectroscopy (IR), spectrophotometry (e.g., UV-visible), or mass spectrometry (MS), or by chromatography such as high performance liquid chromatography (HPLC) or thin layer chromatography (TLC).

Preparation of Compounds can Involve the Protection and Deprotection of Various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Resolution of racemic mixtures of compounds can be carried out by any of numerous methods known in the art. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Resolution of racemic mixtures can also be carried out by elution on a column packed with an optically active resolving agent (e.g., dinitrobenzoylphenylglycine). Suitable elution solvent composition can be determined by one skilled in the art.

The compounds of the invention can be prepared, for example, using the reaction pathways and techniques as described below.

Compounds of the invention can be synthesized according to routine methods by those skilled in the art and as shown in the below Schemes.

Two methods for the synthesis of N-hydroxyamidines (e.g., Example 1) are shown in Scheme 1 where an amide 1-2 is formed from the coupling of an amine (NHR¹A) to an acid 1-1 with a suitable coupling agent, such as HBTU, HATU, DCC or the like. Amide 1-2 is either A) chlorinated with suitable chlorination reagent (such as PCl₅, POCl₃, SO₂Cl₂, or alike) followed by addition of NH₂OH; or B) thionated (e.g., by reaction with Lawesson's reagent) and subsequently S-alkylated (e.g., by treatment with MeI or MeOTf) followed by addition of NH₂OH to afford the desired product 1-3.

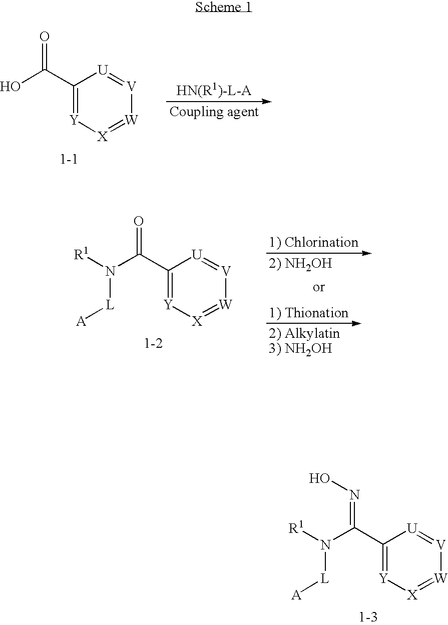

Alternatively, hydroxyamidines, such as 2-4, can be synthesized according to Scheme 2. A halo-heteroaryl derivative 2-1, is substituted with an appropriate amine, NHR^{N1}R^{N2}, and the resulting amino-heteroaryl is converted to the primary hydroxyamidine 2-2 by treatment with NH₂OH. Conversion to the chloroimidate with sodium nitrite and HCl and subsequent addition of an amine, NR^{N1}R^{N2}, gives the desired hydroxyamidines 2-3. Removal of non-hydrogen R^{N1} and/or R^{N2} affords the desired primary amino derivatives 2-4.

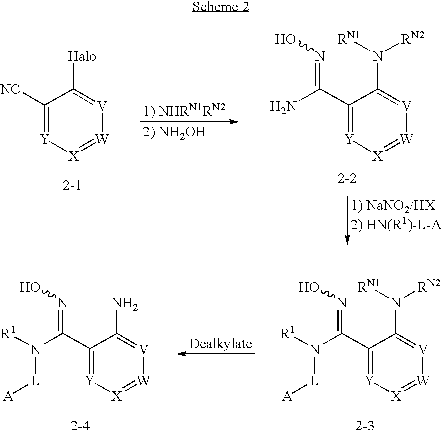

### Methods of Use

Compounds of the invention can modulate activity of the enzyme indoleamine-2,3-dioxygenase (IDO). The term “modulate” is meant to refer to an ability to increase or decrease activity of an enzyme or receptor. Accordingly, compounds of the invention can be used in methods of modulating IDO by contacting the enzyme with any one or more of the compounds or compositions described herein. In some embodiments, compounds of the present invention can act as inhibitors of IDO. In further embodiments, the compounds of the invention can be used to modulate activity of IDO in cell or in an individual in need of modulation of the enzyme by administering a modulating (e.g., inhibiting) amount of a compound of the invention.

The present invention further provides methods of inhibiting the degradation of tryptophan in a system containing cells expressing IDO such as a tissue, living organism, or cell culture. In some embodiments, the present invention provides methods of altering (e.g., increasing) extracellular tryptophan levels in a mammal by administering an effective amount of a compound of composition provided herein. Methods of measuring tryptophan levels and tryptophan degradation are routine in the art.

The present invention further provides methods of inhibiting immunosuppression such as IDO-mediated immunosuppression in a patient by administering to the patient an effective amount of a compound or composition recited herein. IDO-mediated immunosuppression has been associated with, for example, cancers, tumor growth, metastasis, viral infection, viral replication, etc.

The present invention further provides methods of treating diseases associated with activity or expression, including abnormal activity and/or overexpression, of IDO in a person (e.g., patient) by administering to the individual in need of such treatment a therapeutically effective amount or dose of a compound of the present invention or a pharmaceutical composition thereof. Example diseases can include any disease, disorder or condition that is directly or indirectly linked to expression or activity of the IDO enzyme, such as over expression or abnormal activity. An IDO-associated disease can also include any disease, disorder or condition that can be prevented, ameliorated, or cured by modulating enzyme activity. Examples of IDO-associated diseases include cancer, viral infection such as HIV infection, depression, neurodegenerative disorders such as Alzheimer's disease and Huntington's disease, trauma, age-related cataracts, organ transplantation (e.g., organ transplant rejection), and autoimmune diseases including asthma, rheumatoid arthritis, multiple sclerosis, inflammatory bowel disease, psoriasis and systemic lupus erythematosus. Example cancers treatable by the methods herein include cancer of the colon, pancreas, breast, prostate, lung, brain, ovary, cervix, testes, renal, head and neck, lymphoma, leukemia, melanoma, and the like.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the IDO enzyme with a compound of the invention includes the administration of a compound of the present invention to an individual or patient, such as a human, having IDO, as well as, for example, introducing a compound of the invention into a sample containing a cellular or purified preparation containing the IDO enzyme.

As used herein, the term “person” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” is meant to include (1) preventing the disease; for example, preventing a disease, condition or disorder in an individual who may be predisposed to the disease, condition or disorder but does not yet experience or display the pathology or symptomatology of the disease; (2) inhibiting the disease; for example, inhibiting a disease, condition or disorder in an individual who is experiencing or displaying the pathology or symptomatology of the disease, condition or disorder (i.e., arresting further development of the pathology and/or symptomatology); or (3) ameliorating the disease; for example, ameliorating a disease, condition or disorder in an individual who is experiencing or displaying the pathology or symptomatology of the disease, condition or disorder (i.e., reversing the pathology and/or symptomatology).

### Combination Therapy

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2, GM-CSF, etc.), and/or tyrosine kinase inhibitors can be used in combination with the compounds of the present invention for treatment of IDO-associated diseases, disorders or conditions. The agents can be combined with the present compounds in a single dosage form, or the agents can be administered simultaneously or sequentially as separate dosage forms.

Suitable antiviral agents contemplated for use in combination with the compounds of the present invention can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine (ddI); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)—FTC]; beta-LFD4 (also called beta-L-D4C and named beta-L-2′,3′-dicleoxy-5-fluoro-cytidene); DAPD, ((−)beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1549. Other antiviral agents include hydroxyurea, ribavirin, IL-2, IL-12, pentafuside and Yissum Project No. 11607.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (Cytoxan™), ifosfamide, melphalan, chlorambucil, pipobroman, triethylene-melamine, triethylenethiophosphoramine, busulfan, carmustine, lomustine, streptozocin, dacarbazine, and temozolomide.

In the treatment of melanoma, suitable agents for use in combination with the compounds of the present invention include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC; or temozolomide. Compounds according to the invention may also be combined with immunotherapy drugs, including cytokines such as interferon alpha, interleukin 2, and tumor necrosis factor (TNF) in the treatment of melanoma.

Compounds of the invention may also be used in combination with vaccine therapy in the treatment of melanoma. Antimelanoma vaccines are, in some ways, similar to the anti-virus vaccines which are used to prevent diseases caused by viruses such as polio, measles, and mumps. Weakened melanoma cells or parts of melanoma cells called antigens may be injected into a patient to stimulate the body's immune system to destroy melanoma cells.

Melanomas that are confined to the arms or legs may also be treated with a combination of agents including one or more compounds of the invention, using a hyperthermic isolated limb perfusion technique. This treatment protocol temporarily separates the circulation of the involved limb from the rest of the body and injects high doses of chemotherapy into the artery feeding the limb, thus providing high doses to the area of the tumor without exposing internal organs to these doses that might otherwise cause severe side effects. Usually the fluid is warmed to 102° to 104° F. Melphalan is the drug most often used in this chemotherapy procedure. This can be given with another agent called tumor necrosis factor (TNF) (see section on cytokines).

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, anti-tumor antibiotics, enzymes, lymphokines and epipodophyllotoxins) such as vinblastine, vincristine, vindesine, bleomycin, dactinomycin, daunorubicin, doxorubicin, epirubicin, idarubicin, araC, paclitaxel (TAXOL™), mithramycin, deoxycoformycin, mitomycin-C, L-asparaginase, interferons (especially IFN-a), etoposide, and teniposide.

Other cytotoxic agents include navelbene, CPT-11, anastrazole, letrazole, capecitabine, reloxafine, cyclophosphamide, ifosamide, and droloxafine.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (Herceptin), antibodies to costimulatory molecules such as CTLA-4, 4-1BB and PD-1, or antibodies to cytokines (IL-10, TGF-β, etc.).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, N.J.), the disclosure of which is incorporated herein by reference as if set forth in its entirety.

### Pharmaceutical Formulations and Dosage Forms

When employed as pharmaceuticals, the compounds of the invention can be administered in the form of pharmaceutical compositions which is a combination of a compound of the invention and a pharmaceutically acceptable carrier. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the invention may be milled using known milling procedures such as wet milling to obtain a particle size appropriate for tablet formation and for other formulation types. Finely divided (nanoparticulate) preparations of the compounds of the invention can be prepared by processes known in the art, for example see International Patent Pub. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 100 mg, more usually from about 10 mg to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, from about 0.1 mg to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be from about 3 to about 11, or from about 5 to about 9, or from about 7 to about 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing from about 0.1% to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds of the invention can also be formulated in combination with one or more additional active ingredients which can include any pharmaceutical agent such as anti-viral agents, vaccines, antibodies, immune enhancers, immune suppressants, anti-inflammatory agents and the like.

### Labeled Compounds and Assay Methods

Another aspect of the present invention relates to fluorescent dye, spin label, heavy metal or radio-labeled compounds of the invention that would be useful not only in imaging but also in assays, both in vitro and in vivo, for localizing and quantitating the IDO enzyme in tissue samples, including human, and for identifying IDO enzyme ligands by inhibition binding of a labeled compound. Accordingly, the present invention includes IDO enzyme assays that contain such labeled compounds.

The present invention further includes isotopically-labeled compounds of Formula I.

An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro IDO enzyme labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled” or “labeled compound” is a compound that has incorporated at least one radionuclide. In some embodiments the radionuclide is selected from the group consisting of ³H, ¹⁴C, ¹²⁵I, ³⁵S and ³²Br.

Synthetic methods for incorporating radio-isotopes into organic compounds are applicable to compounds of the invention and are well known in the art.

A radio-labeled compound of the invention can be used in a screening assay to identify/evaluate compounds. In general terms, a newly synthesized or identified compound (i.e., test compound) can be evaluated for its ability to reduce binding of the radio-labeled compound of the invention to the IDO enzyme. Accordingly, the ability of a test compound to compete with the radio-labeled compound for binding to the IDO enzyme directly correlates to its binding affinity.

### Kits

The present invention also includes pharmaceutical kits useful, for example, in the treatment or prevention of IDO-associated diseases or disorders, obesity, diabetes and other diseases referred to herein which include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

The compounds of the Examples were found to be inhibitors of IDO according to the assay of Example A an optionally one or more of the other assays provided herein. In some instances where the compounds of the examples were isolated by preparative HPLC in the presence of trifluoroacetic acid (TFA) or other acid, the compound may have been obtained as the corresponding salt.

### EXAMPLES

### Example 1

### NA-Hydroxy-3-[(4-methoxybenzyl)amino]-N-[3-(trifluoromethyl)phenyl]-pyrazine-2-carboximidamide

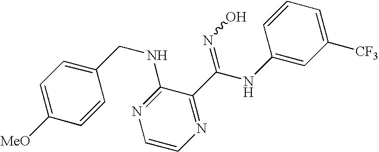

### Step A: 3-[(4-Methoxybenzyl)amino]pyrazine-2-carbonitrile

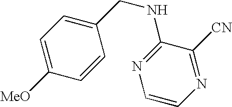

To a solution of 3-chloropyrazine-2-carbonitrile (500 mg, 3.6 mmol) in dichloromethane (DCM) (4.0 mL) was added, 4-methoxy-benzenemethanamine (470 μL, 0.0036 mol), N,N-diisopropylethylamine (620 μL, 0.0036 mol) and the resulting mixture was stirred at room temperature (25° C.) for 5 h. The crude residue was purified by flash column chromatography to yield the desired product (650 mg, 75%). MF=C₁₃H₁₂N₄O; LCMS calculated for C₁₃H₁₃N₄O(M+H)⁺: m/z=241.1.

### Step B: N′-Hydroxy-3-[(4-methoxybenzyl)amino]pyrazine-2-carboximidamide

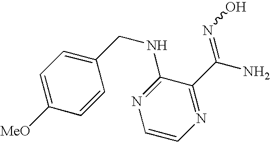

To a solution of 3-[(4-methoxybenzyl)amino]pyrazine-2-carbonitrile (0.50 g, 0.0021 mol) in methanol (8.0 mL) was added 20 M of hydroxylamine in water (3.0 mL). The resulting solution was allowed to stir for 10 min. and then was cooled in an ice bath for 20 min. A solid precipitate formed and was filtered off, washed with cold water and dried to afford the desired product as a white solid (560 mg, 98%). MF=C₁₃H₁₅N₅O₂; LCMS calculated for C₁₃H₁₆N₅O₂(M+H)⁺: m/z=274.1.

### Step C: N-Hydroxy-3-[(4-methoxybenzyl)amino]pyrazine-2-carboximidoyl chloride

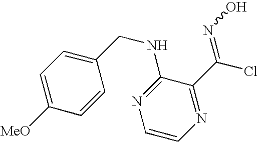

To a solution of N′-hydroxy-3-[(4-methoxybenzyl)amino]pyrazine-2-carboximidamide (50 mg, 0.18 mmol) in water (1.5 mL, 0.083 mol) and hydrogen chloride (0.50 mL, 0.016 mol) was added dimethyl sulfoxide (1.0 mL) at 0° C. To the rapidly stirring solution was added a solution of sodium nitrite (43 mg, 0.63 mmol) in water (0.50 mL) over 5 minutes. The crude material was purified by preparative HPLC to afford the desired product (47 mg, 88%). MF=C₁₃H₁₃ClN₄O₂; LCMS calculated for C₁₃H₁₄ClN₄O₂(M+H)⁺: m/z=293.1.

### Step D: N′-Hydroxy-3-[(4-methoxybenzyl)amino]-N-[3-(trifluoromethyl)phenyl]pyrazine-2-carboximidamide

To a solution of N-hydroxy-3-[(4-methoxybenzyl)amino]pyrazine-2-carboximidoyl chloride (10.0 mg, 0.0342 mmol) in ethanol (1.5 mL) was added 3-(trifluoromethyl)benzenamine, (6.6 mg, 0.041 mmol) followed by N,N-diisopropylethylamine (7.1 μL, 0.041 mmol). The resulting solution was stirred at room temperature for 1 h. The crude product of the reaction was purified by preparative HPLC to afford the desired product (12 mg, 84%). MF=C₂₀H₁₈F₃N₅O₂; LCMS calculated for C₂₀H₁₉F₃N₅O₂(M+H)⁺: m/z=418.1. ¹H NMR (400 MHz, CD₃OD) δ: 8.03 (s, 1H), 7.78 (s, 1H), 7.35 (m, 1H), 7.20 (m, 3H), 7.03 (m, 2H), 6.88 (m, 2H), 4.60 (s, 2H), 3.79 (s, 3H).

### Example 2

### 3-Amino-N′-hydroxy-N-[3-(trifluoromethyl)phenyl]pyrazine-2-carboximidamide

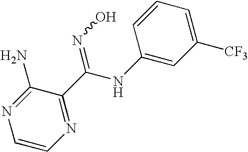

N-[3-(Trifluoromethyl)phenyl]-N′-hydroxy-3-[(4-methoxybenzyl)amino]pyrazine-2-carboximidamide (20 mg, 0.04 mmol) was dissolved in trifluoroacetic acid (2.0 mL) and the resulting solution was stirred for 4 h. The crude product was purified by preparative HPLC to afford the desired product (8 mg, 55%). MF=C₁₂H₁₀F₃N₅O; LCMS calculated for C₁₂H₁₁F₃N₅O(M+H)⁺: m/z=298.1. ¹H NMR (400 MHz, DMSO-d₆) δ: 11.17 (S, 1H), 8.70 (s, 1H), 8.02 (s, 1H), 7.77 (s, 1H), 7.32 (t, 1H), 7.20 (bs, 2H), 7.11 (d, 1H), 6.96 (m, 2H).

### Example 3

### N-(3-Chloro-4-fluorophenyl)-N′-hydroxybiphenyl-4-carboximidamide

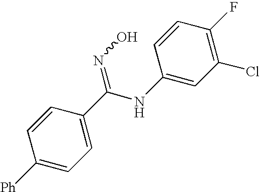

### Step A: N-(3-Chloro-4-fluorophenyl)biphenyl-4-carboxamide

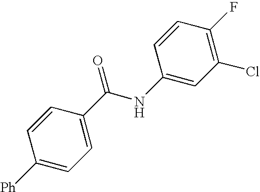

To a solution of biphenyl-4-carboxylic acid (41 mg, 0.206 mmol) in DCM (1.5 mL) was added bromotris(pyrrolydino)phosphonium hexafluorophosphate (96.1 mg, 0.206 mmol), followed by N,N-diisopropylethylamine (72 μL, 0.41 mmol) and 3-chloro-4-fluoroaniline (30.0 mg, 0.206 mmol). The resulting mixture was stirred for 3 h and then the volatiles were removed under vacuum to provide a crude product, which was purified by preparative HPLC to afford the desired product (42 mg, 71%). MF=C₁₉H₁₃ClF NO; LCMS calculated for C₁₉H₁₄ClFNO (M+H)⁺: m/z=326.

### Step B: N-(3-Chloro-4-fluorophenyl)biphenyl-4-carbothioamide

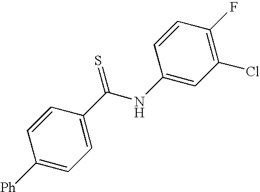

To solution of N-(3-chloro-4-fluorophenyl)biphenyl-4-carboxamide (42 mg, 0.13 mmol) in toluene (1.5 mL) was added 2,4-bis(4-methoxyphenyl)-2,4-dithioxo-1,3,2,4-dithiadiphosphetane (112 mg, 0.28 mmol). The resulting mixture was sealed and heated at 100° C. overnight. The volatiles were removed under vacuum to provide a crude product, which was purified by preparative LCMS to afford the desired product (17 mg, 38%). MF=C₁₉H₁₃ClFNS; LCMS calculated for C₁₉H₁₄ClFNS (M+H)⁺: m/z=342.

### Step C: N-(3-Chloro-4-fluorophenyl)-N′-hydroxybiphenyl-4-carboximidamide

To a solution of N-(3-chloro-4-fluorophenyl)-4-phenylbenzenecarbothioamide (17 mg, 0.05 mmol) in DCM (1.0 mL) was added methyl trifluoromethanesulfonate (5 μL, 0.05 mmol) and N,N-diisopropylethylamine (11 μL, 0.062 mol). The resulting solution was stirred overnight. The volatiles were removed under vacuum and to the resulting residue was added a solution of hydroxylamine in water (100 μL of 15 M) and ethanol (0.8 mL) and the resulting mixture was stirred overnight. The volatiles were removed under vacuum and the resulting residue was purified by preparative HPLC to afford the desired product (8 mg). MF=C₁₉H₁₄ClFN₂O; LCMS calculated for C₁₉H₁₅ClFN₂O (M+H)⁺: m/z=341.1.

Further example compounds of the invention are provided in Table 1 below.

TABLE 1

| Ex. | | | Prep. | MS |
|---|---|---|---|---|
| No. | Structure | Name | Method | (M + 1) |
| 4 | 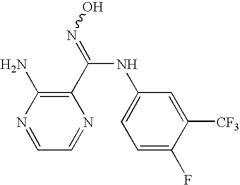 | 3-amino-N′-hydroxy-N-[3-(tri-fluoromethyl), 4-fluorophenyl]-pyrazine-2-carboximidamide | Example 2 | 316 |
| 5 | 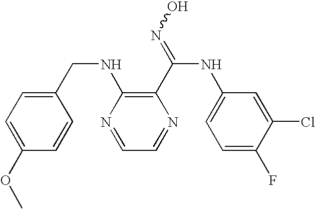 | N′-hydroxy-3-[(4-methoxybenzyl)-amino]-N-[3-(trifluoromethyl)-4-fluorophenyl]pyrazine-2-carbox-imidamide | Example 1 | 402 |
| 6 | 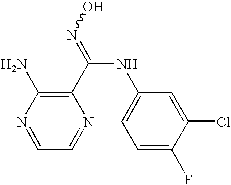 | 3-amino-N′-hydroxy-N-[3-chloro,4-fluorophenyl]pyrazine-2-carbox-imidamide | Example 2 | 282 |
| 7 | 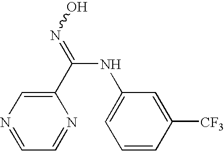 | N′-hydroxy-N-[3-(trifluoromethyl)-phenyl]pyrazine-2-carbox-imidamide | Example 1 | 283 |
| 8 | 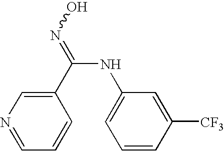 | N′-hydroxy-N-[3-(trifluoromethyl)-phenyl]pyridine-3-carbox-imidamide | Example 1 | 282 |
| 9 | 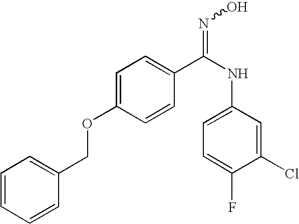 | 4-(benzyloxy)-N-(3-chloro-4-fluorophenyl)-N′-hydroxybenzene-carboximidamide | Example 3 | 371 |
| 10 | 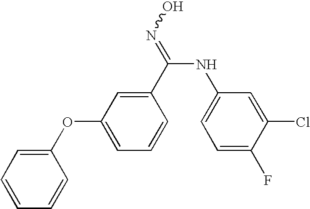 | N-(3-chloro-4-fluorophenyl)-N′-hydroxy-3-phenoxybenzene-carboximidamide | Example 3 | 357 |
| 11 | 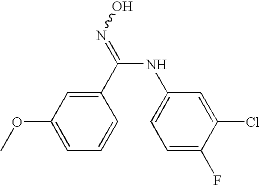 | N-(3-chloro-4-fluorophenyl)-N′-hydroxy-3-methoxybenzene-carboximidamide | Example 3 | 295 |
| 12 | 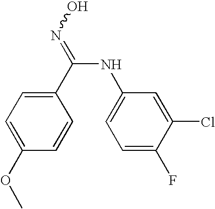 | N-(3-chloro-4-fluorophenyl)-N′-hydroxy-4-methoxybenzenecarbox-imidamide | Example 3 | 295 |
| 13 | 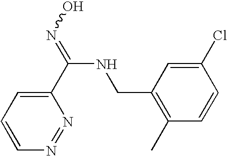 | N-(5-chloro-2-methylbenzyl)-N′-hydroxypyridazine-3-carboximidamide | Example 3 | 277 |

### NMR or Example 4

¹H NMR (400 MHz, CD₃OD) δ: 7.97 (s, 1H), 7.85 (s, 1H), 7.10 (m, 3H).

### NMR of Example 5

¹H NMR (400 MHz, CD₃OD) δ: 78.05 (s, 1H), 7.78 (s, 1H), 7.21 (d, 3H), 7.0 (t, 1H), 6.83 (m, 2H), 6.72 (m, 1H), 4.58 (s, 2H), 3.79 (s, 3H).

### NMR of Example 6

¹H NMR (400 MHz, CD₃OD) δ: 7.97 (s, 1H), 7.83 (s, 1H), 7.05 (t, 1H), 6.93, (m, 1H), 6.78 (m, 1H).

### NMR of Example 7

¹H NMR (400 MHz, CD₃OD) δ: 9.00 (s, 1H), 8.60 (s, 1H), 8.51 (s, 1H), 7.30 (m, 1H), 7.17 (m, 1H), 7.01 (s, 1H), 6.94 (m, 1H).

### NMR of Example 9

¹H NMR (400 MHz, CD₃OD) δ: 7.38 (m, 7H), 7.03 (m, 4H), 6.80 (m, 1H), 5.11 (s, 3H).

### NMR of Example 10

¹H NMR (400 MHz, CD₃OD) δ: 7.45 (t, 1H), 7.28 (m, 3H), 7.12 (m, 3H), 7.00 (d, 1H), 6.80 (m, 4H).

### NMR of Example 11

¹H NMR (400 MHz, CD₃OD) δ: 7.39 (m, 1H), 7.23 (m, 1H), 7.15 (m, 2H), 7.01 (m, 3H), 3.77 (s, 3H).

### NMR of Example 13

¹H NMR (400 MHz, DMSO-d₆) δ: 10.35 (s, 1H), 9.20 (d, J=4.7 Hz, 1H), 7.78 (d, J=6.4 Hz, 1H), 7.65 (dd, J=4.9 Hz, 8.7 Hz, 1H), 7.14 (m, 1H), 7.09 (m, 1H), 7.07 (s, 1H), 6.84 (m, 1H), 4.55 (s, 2H), 2.08 (s, 3H).

### Example A

### Human indoleamine 2,3-dioxygenasae (IDO) enzyme assay

Human indoleamine 2,3-dioxygenasae (IDO) with an N-terminal His tag was expressed in E. coli and purified to homogeneity. IDO catalyzes the oxidative cleavage of the pyrrole ring of the indole nucleus of tryptophan to yield N′-formylkynurenine. The assays were performed at room temperature as described in the literature using 95 nM IDO and 2 mM D-Trp in the presence of 20 mM ascorbate, 5 μM methylene blue and 0.2 mg/mL catalase in 50 mM potassium phosphate buffer (pH 6.5). The initial reaction rates were recorded by continuously following the absorbance increase at 321 nm due to the formation of N′-formlylkynurenine. See: Sono, M., Taniguchi, T., Watanabe, Y., and Hayaishi, O. (1980) J. Biol. Chem. 255, 1339-1345. Compounds of the invention were found to be inhibitors of IDO according to this assay. Data is provided below in Table 2. The symbol “+” indicates IC₅₀<1000 μM. The symbol “++” indicates 1000≦IC₅₀≦10,000 μM. The symbol “+++” indicates IC₅₀>10,000 μM

TABLE 2

| Ex. No. | IC₅₀ (μM) |
|---|---|
| 1 | +++ |
| 2 | +++ |
| 3 | ++ |
| 4 | +++ |
| 5 | +++ |
| 6 | +++ |
| 7 | +++ |
| 8 | +++ |
| 9 | ++ |
| 10 | +++ |
| 11 | ++ |
| 12 | ++ |
| 13 | +++ |

### Example B

### Determination of Inhibitor Activity in HeLa Cell-Based indoleamine 2,3-dioxygenase (IDO)/Kynurenine Assay

HeLa cells (#CCL-2) were obtained from the American Type Tissue Culture Collection (ATCC, Manassas, Va.) and routinely maintained in minimum essential medium (eagle) with 2 mM L-glutamine and Earle's BSS adjusted to contain 1.5 g/L sodium bicarbonate, 0.1 mM non-essential amino acids, 1 mM sodium pyruvate and 10% fetal bovine serum (all from Invitrogen). Cells were kept at 37° C. in a humidified incubator supplied with 5% CO₂. The assay was performed as follows: HeLa cells were seeded in a 96 well culture plate at a density of 5×10³ per well and grown overnight. On the next day, IFN-γ (50 ng/mL final concentration) and serial dilutions of compounds (in total volume of 200 μL culture medium) were added into cells. After 48 hours of incubation, 140 μL of the supernatant per well was transferred to a new 96 well plate. 10 μL of 6.1 N trichloroacetic acid (#T0699, Sigma) was mixed into each well and incubated at 50° C. for 30 min to hydrolyze N-formylkynurenine produced by indoleamine 2,3-dioxygenase to kynurenine. The reaction mixture was then centrifuged for 10 min at 2500 rpm to remove sediments. 100 μL of the supernatant per well was transferred to another 96 well plate and mixed with 100 μl of 2% (w/v) p-dimethylaminobenzaldehyde (#15647-7, Sigma-Aldrich) in acetic acid. The yellow color derived from Kynurenine was measured at 480 nm using a SPECTRAmax 250 microplate reader (Molecular Devices). L-kynurenine (#K8625, Sigma) was used as standard. The standards (240, 120, 60, 30, 15, 7.5, 3.75, 1.87 μM) were prepared in 100 μL culture media and mixed with equal volume of 2% (w/v) p-dimethylaminobenzaldehyde. The percent inhibition at individual concentrations was determined and the average values of duplicates were obtained. The data was analyzed by using nonlinear regression to generate IC₅₀ values (Prism Graphpad). See: Takikawa O, et al. (1988). Mechanism of interferon-gamma action. Characterization of indoleamine 2,3-dioxygenase in cultured human cells induced by interferon-gamma and evaluation of the enzyme-mediated tryptophan degradation in its anticellular activity. J. Biol. Chem. 263(4):2041-8. Example compounds that were tested according to this assay were found to have IC₅₀ values ranging from 1800 to 7000 μM.

### Example C

### Determination of Effect of IDO Inhibitors on T Cell Proliferation that is Suppressed by IDO-Expressing Dendritic Cells

Monocytes are collected from human peripheral mononuclear cells by leukophoresis. Monocytes are then seeded at a density of 1×10⁶ cells/well in a 96 well plate, using RPMI 1640 medium supplemented with 10% fetal bovine serum and 2 mM L-glutamine (all from Invitrogen). Adherent cells are retained on the plate after overnight culture at 37° C. Adherent monocytes are then stimulated for 5-7 days with 100 ng/mL GM-CSF (# 300-03, PeproTech) and 250 ng/mL IL-4 (#200-04, PeproTech), followed by activation with 5 μg/mL LPS from Salmonella typhimurium (#437650, Sigma) and 50 ng/mL IFN-γ (# 285-IF, R&D Systems) for additional 2 days to induce dendritic cell maturation.

After dendritic cell activation, the medium is replaced with completed RPMI 1640 supplemented with 100-200 U/mL IL-2 (#CYT-209, ProSpec-Tany TechnoGene) and 100 ng/mL anti-CD3 antibody (#555336, PharMingen), T cells (2-3×10⁵ cells/well), and serial dilutions of IDO compounds. After incubation for 2 more days, T cell proliferation is measured by BrdU incorporation assay, using a colorimetric Cell Proliferation ELISA kit per manufacturer's instruction (#1647229, Roche Molecular Biochemicals). Cells are continuously cultured for 16-18 hrs in presence of 10 μM BrdU labeling solution. Then, the labeling medium is removed, and 200 μL FixDenat per well is added to the cells and incubated for 30 minutes at room temperature. The FixDenat solution is removed and 100 μL/well anti-BrdU-POD antibody conjugate working solution was added. The reaction is carried out for 90 minutes at room temperature. The antibody conjugate is then removed, and cells are rinsed three times with 200 μL/well washing solution. Finally, 100 μL/well of substrate solution is added and the results are obtained using a microplate reader (Spectra Max PLUS, Molecular Devices) during color development. Multiple readings at various time points are obtained to ensure the data was within the linear range. The data is routinely obtained from replicated experiments, and appropriate controls were included. See: Terness P, et al. (2002). Inhibition of allogeneic T cell proliferation by indoleamine 2,3-dioxygenase-expressing dendritic cells: mediation of suppression by tryptophan metabolites. J. Exp. Med. 196(4):447-57; and Hwu P, et al. (2000). Indoleamine 2,3-dioxygenase production by human dendritic cells results in the inhibition of T cell proliferation. J. Immunol. 164(7):3596-9.

### Example D

### In Vivo Testing of IDO Inhibitors for Antitumor Activity

In vivo anti-tumor efficacy can be tested using modified tumor allograft/xenograft protocols. For instance, it has been described in the literature that IDO inhibition can syngerize with cytotoxic chemotherapy in immune-competent mice (Muller, A. J., et al). This synergy was shown to be dependent on T-cells by comparison of the synergistic effects of an investigational IDO inhibitor in murine tumor xenograft models (e.g. B16 and related variants, CT-26, LLC) grown in immune competent syngenic mice to that observed in syngenic mice treated with neutralizing anti-CD4 antibodies, or the same tumors grown in immune-compromised mice (e.g. nu/nu).

The concept of differential anti-tumor effects in immune-competent versus immune-compromised mice may also permit testing of investigational IDO inhibitors as single agents. For instance, LLC tumors grow well in their syngenic host strain, C57Bl/6. However, if these mice are treated with the IDO inhibitor 1-MT (versus placebo) the formation of tumors is markedly delayed, implying that IDO inhibition was growth inhibitory (Friberg, M., et al). Following this logic, one can examine the efficacy of IDO inhibition in the LLC xenograft tumor model grown in C57Bl/6 immune competent mice and compare that to the effects of IDO inhibitors on LLC tumor growth in nude or SCID mice (or C57Bl/6 mice treated with antibodies that neutralize T-cell activity). As the effects of relieving the tumor-mediated immune suppressive activity of IDO will likely differ depending on the immunogenic potential of different tumor models, genetic modifications can be made to the tumor cells to increase their immunogenic potential. For instance, expression of GM-CSF in B16.F10 cells increases their immunogenic potential (Dranoff, G., et al). As such, in some tumor models (e.g. B16.F10) one can generate [poly]clones that express immune stimulatory proteins such as GM-CSF and test the growth inhibitory effects of IDO inhibitors against tumors established from these tumor cells in both immune-competent and compromised mice.

A third avenue for assessing the efficacy of IDO inhibitors in vivo employs ‘preimmunization’ murine tumor allograft/xenograft models. In these models, immune-competent mice are sensitized to a specific tumor antigen or antigens to mimic a therapeutic anti-tumor vaccination. This primes the mice for an anti-tumor response mediated by the immune system when mice are subsequently challenged with murine tumor cell lines (possessing similar tumor antigens to those used for immunization) in xenograft experiments. Expression of IDO has been shown to blunt the anti-tumor response and allow xenografts to grow more rapidly. Importantly, the growth of tumors in this model is inhibited by the IDO inhibitor 1-MT (Uyttenhove, C., et al). This model is particularly attractive as IDO activity is permissive for P815 tumor growth and specific inhibition of IDO should therefore growth inhibitory.

Lastly, therapeutic immunization may be used to evaluate the impact of IDO inhibitors in vivo. For example, it has been demonstrated using B16-BL6 cells that one can challenge Blk/6 mice with an intravenous injection of tumor cells followed by treatment with a well characterized immunogenic peptide (e.g. TRP-2; SVYDFFVWL) expressed by the tumor cells (Ji, et al., J. Immunol, 2005, 175: 1456-63). Importantly, immune system modifiers, such as anti-CTL-4 antibody, can improve responses to such therapeutic immunizations. The impact of IDO inhibitors may be evaluated in a similar manner—tumor peptide immunization with or without IDO inhibitor. Efficacy is assess by animal survival (time to morbidity) or by the measurement of tumor metastases to the lungs and/or other organs at defined timepoints.

In any/all of the above mentioned models, it may also be possible to directly and/or indirectly measure the number and/or activity of tumor reactive immune cells. Methods for measuring the number and/or activity of tumor reactive immune cells are well established and can be performed using techniques familiar to those schooled in the art (Current Protocols in Immunology, vol 4, Coligan, J. E., et al; Immunotherapy of Cancer, Human Press, 2006, Disis, M. L. and references therein). Conceptually, a reduction in the immune suppressive effects of IDO may result in increased numbers or reactivity of tumor specific immune cells. Further, IDO inhibition may further increase the number or reactivity of tumor reactive immune cells when combined with other therapeutics, for example chemotherapeutics and/or immune modulators (e.g. anti-CTLA4 antibody).

All allograft/xenograft experiments can be performed using standard tumor techniques (reviewed by Corbett, et al). The cloning and introduction of genes (e.g. IDO, GM-CSF) into tumor cell lines, can be performed using techniques familiar to those schooled in the art (reviewed in Sambrook, J, et al.). See: Corbett, T., Polin, L., et al. In vivo methods for screening and preclinical testing. Cancer Drug Discovery and Development: Anticancer Drug Development Guide: Preclinical Screening, Clinical Trials, and Approval, 2^{nd} Ed. Teicher, B. A. and Andrews, P. A., Gumana Press Inc., Totowa, N.J., 2004; Dranoff, G., Jaffee, E., et al. Vaccination with irradiated tumor cells engineered to secrete murine granulocyte-macrophage colony-stimulating factor stimulates potent, specific, and long-lasting anti-tumor immunity. Proc. Natl. Acad. Sci, USA. 90:3539-3543, 1993; Friberg, M., Jennings, R., et al. Indoleamine 2,3-dioxygenase contributes to tumor cell evasion of T cell-mediated rejection. Int. J. Cancer: 101:151-155, 2002; Muller, A. J., DuHadaway, J. B., et al. Inhibition of indoleamine 2,3-dioxygenase, an immunoregulatory target of the cancer suppression gene Bin1, potentiates cancer chemotherapy. Nat. Med. 11:312-319, 2005; Sambrook, J, Russel, D. Molecular Cloning: A laboratory Manual (3rd edition). Cold Spring Harbor Laboratory Press. Cold Spring Harbor, N.Y., USA. 2001; and Uyttenhove, C., Pilotte, L., et al. Evidence for a tumoral immune resistance mechanism based on tryptophan degradation by indoleamine 2,3-dioxygenase. Nat. Med. 9:1269-1274, 2003.

### Example E

### In Vivo Testing of IDO Inhibitors in Human Immunodeficiency Virus-1 (HIV-1) Encephalitis Model

1. Cell Isolation and Viral Infection

Monocytes and PBL can be obtained by countercurrent centrifugal elutriation of leukopheresis packs from HIV-1, 2 and hepatitis B seronegative donors. Monocytes are cultivated in suspension culture using Teflon flasks in Dulbecco's Modified Eagle's Medium (DMEM, Sigma-Aldrich) supplemented with 10% heat-inactivated pooled human serum, 1% glutamine, 50 μg/mL gentamicin, 10 μg/mL ciprofloxacin (Sigma), and 1000 U/mL highly purified recombinant human macrophage colony stimulating factor. After seven days in culture, MDM are infected with HIV-1_{ADA} at multiplicity of infection of 0.01.

2. Hu-PBL-NOD/SCID HIVE Mice

Four-wk old male NOD/C.B-17 SCID mice can be purchased (Jackson Laboratory). Animals are maintained in sterile microisolator cages under pathogen-free conditions. All animals are injected intraperitoneally with rat anti-CD122 (0.25 mg/mouse) three days before PBL transplantation and twice with rabbit asialo-GM1 antibodies (0.2 mg/mouse) (Wako) one day before and three days after PBL injection (20×10⁶ cells/mouse). HIV-1_{ADA}-infected MDM (3×10⁵ cells in 10 μL) are injected intracranially (i.c.) eight days following PBL reconstitution generating hu-PBL-NOD/SCID HIVE mice. Immediately following i.c. injection of HIV-1 infected MDM the hu-PBL-NOD/SCID HIVE mice are subcutaneously (s.c) implanted with control (vehicle) or compound pellets (14 or 28 day slow release, Innovative Research). Initial experiments are designed to confirm the induction of virus-specific CTL in the hu PBL-NOD/SCID HIVE animals treated with IDO compounds. This is confirmed by tetramer staining and neuropathologic analyses of MDM elimination from the brain tissue. Then, the experiment is designed to analyze human lymphocyte reconstitution, humoral immune responses, and neuropathological alterations. In these experiments, animals are bled on day 7 and sacrificed at 14 and 21 days after i.c. injection of human MDM. Blood collected in EDTA-containing tubes is used for flow cytometry and plasma is used for detection of HIV-1 p24 using ELISA (Beckman Coulter™). HIV-1-specific antibodies are detected by Western blot tests according to the manufacturer instructions (Cambridge Biotech HIV-1 Western blot kit, Calypte Biomedical). Similar amount of virus-specific antibodies are detected in control and compound-treated animals. A total of three independent experiments can be performed using three different human leukocyte donors.

3. FACScan of Peripheral Blood and Spleen in hu PBL-NOD/SCID HIVE mice

Two-color FACS analysis can be performed on peripheral blood at wk 1-3 and splenocytes at wk 2 and 3 after i.c. injection of human MDM. Cells are incubated with fluorochrome-conjugated monoclonal Abs (mAbs) to human CD4, CD8, CD56, CD3, IFN-γ (eBioscience) for 30 min at 4° C. To evaluate the cellular immune response, IFN-γ intracellular staining is performed in combination with anti-human CD8 and FITC-conjugated anti-mouse CD45 to exclude murine cells. To determine the Ag-specific CTL, allophycocyanin-conjugated tetramer staining for HIV-1^{gag} (p17 (aa77-85) SLYNTVATL, SL-9) and HIV-1^{pol} [(aa476-485) ILKEPVHGV, IL-9] is performed on phytohemaglutinin/interleukin-2 (PHA/IL-2)-stimulated splenocytes. Cells are stained following the recommendation of the NIH/National Institute of Allergy and Infections Disease, National Tetramer Core Facilities. Data were analyzed with a FACS CALIBUR™ using CellQuest software (Becton Dickinson Immunocytometry System).

4. Histopathology and Image Analyses

Brain tissue is collected at days 14 and 21 after i.c. injection of MDM, fixed in 4% phosphate-buffered paraformaldehyde and embedded in paraffin or frozen at −80° C. for later use. Coronal sections from the embedded blocks are cut in order to identify the injection site. For each mouse, 30-100 (5-μm-thick) serial sections are cut from the human MDM injection site and 3-7 slides (10 sections apart) are analyzed. Brain sections are deparaffinized with xylene and hydrated in gradient alcohols. Immunohistochemical staining follows a basic indirect protocol, using antigen retrieval by heating to 95° C. in 0.01 mol/L citrate buffer for 30 min for antigen retrieval. To identify human cells in mouse brains, mAb to vimentin (1:50, clone 3B4, Dako Corporation), which identifies all human leukocytes is used. Human MDM and CD8⁺ lymphocytes are detected with CD68 (1:50 dilution, clone KP 1) and CD8 (1:50 dilution, clone 144B) antibodies, respectively. Virus-infected cells are labeled with mAb to HIV-1 p24 (1:10, clone Kal-1, all from Dako). Reactive murine microglial cells are detected with Jba-1 antibody (1:500, Wako). Expression of human IDO (huIDO) is visualized with Abs obtained from the Department of Cell Pharmacology, Central Research Institute, Graduate School of Medicine, Hokkaido University, Sapporo, Japan. Primary antibodies are detected with the appropriate biotinylated secondary antibodies and visualized with avidin-biotin complexes (Vectastain Elite ABC kit, Vector Laboratories) and horseradish peroxidase (HRP) coupled dextran polymer (EnVision, Dako Corporation). Immunostained sections are counterstained with Mayer's hematoxylin. Sections from which primary antibody is deleted or irrelevant IgG isotype is incorporated served as controls. Two independent observers in a blinded fashion count the numbers of CD8⁺ lymphocytes, CD68⁺ MDM and HIV-1 p24⁺ cells in each section from each mouse. Light microscopic examination is performed with a Nikon Eclipse 800 microscope (Nikon Instruments Inc). Semi-quantitative analysis for Iba1 (percentage of area occupied by immunostaining) is carried out by computer-assisted image analysis (Image-Pro®Plus, Media Cybernetics) as previously described.

5. Statistical Analysis

Data can be analyzed using Prism (Graph Pad) with Student t-test for comparisons and ANOVA. P-values <0.05 were considered significant.

6. Reference

Poluektova L Y, Munn D H, Persidsky Y, and Gendelman H E (2002). Generation of cytotoxic T cells against virus-infected human brain macrophages in a murine model of HIV-1 encephalitis. J. Immunol. 168(8):3941-9.

Various modifications of the invention, in addition to those described herein, will be apparent to those skilled in the art from the foregoing description. Such modifications are also intended to fall within the scope of the appended claims. Each reference, including all patents, patent applications, and publications, cited in the present application is incorporated herein by reference in its entirety.

What is claimed is:

1. A compound of Formula Ia: 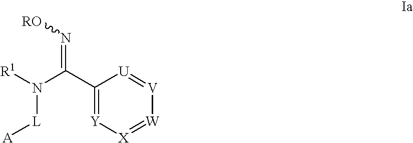
or pharmaceutically acceptable salt thereof, wherein:
U, V, W, X and Y are independently selected from N and CR^{x}, wherein 0, 1, 2 or 3 of U, V, W, X and Y are N, and the remainder of U, V, W, X and Y are CR^{x};
L is a bond, C₁₋₆ alkylene, C₂₋₆ alkenylene, C₂₋₆ alkynylene, (C₁₋₆ alkylene)ᵣ-O—(C₁₋₆ alkylene)ₛ, (C₁₋₆ alkylene)ᵣ-S—(C₁₋₆ alkylene)ₛ, (C₁₋₆ alkylene)ᵣ-NR^{j}-(C₁₋₆ alkylene)ₛ, (C₁₋₆ alkylene)ᵣ-CO—(C₁₋₆ alkylene)ₛ, (C₁₋₆ alkylene)ᵣ-COO—(C₁₋₆ alkylene)ₛ, (C₁₋₆ alkylene)ᵣ-CONR^{j}—(C₁₋₆ alkylene)ₛ, (C₁₋₆ alkylene)ᵣ-SO—(C₁₋₆ alkylene)ₛ, (C₁₋₆ alkylene)ᵣ-SO₂—(C₁₋₆ alkylene)ₛ, (C₁₋₆ alkylene)ᵣ-SONR^{j}—(C₁₋₆ alkylene)ₛ, (C₁₋₆ alkylene)ᵣ-SO₂NR^{j}—(C₁₋₆ alkylene)ₛ, or (C₁₋₆ alkylene)ᵣ-NR^{j}CONR^{k}—(C₁₋₆ alkylene)ₛ, wherein each of the C₁₋₆ alkylene, C₂₋₆ alkenylene, and C₂₋₆ alkynylene is optionally substituted by 1, 2, or 3 substituents independently selected from halo, CN, NO₂, N₃, SCN, OH, C₁₋₆ alkyl, C₁₋₆ haloalkyl, C₂₋₈ alkoxyalkyl, C₁₋₆ alkoxy, C₁₋₆ haloalkoxy, amino, C₁₋₆ alkylamino, and C₂₋₈ dialkylamino;
A is aryl, cycloalkyl, heteroaryl, or heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₁₋₆ hydroxyalkyl, C₁₋₆ cyanoalkyl, pentahalosulfanyl, Cy, CN, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, P(R^{f})₂, P(OR^{e})₂, P(O)R^{e}R^{f}, P(O)OR^{e}OR^{f}S(O)R^{e}, S(O)NR^{c}R^{d}, S(O)₂R^{b}, NR^{c}S(O)₂R^{b}, and S(O)₂NR^{c}R^{d}, wherein said C₁₋₆ alkyl, C₂₋₆ alkenyl, and C₂₋₆ alkynyl is optionally substituted with 1, 2, or 3 substituents independently selected from Cy, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, P(R^{f})₂, P(OR^{e})₂, P(O)R^{e}R^{f}, P(O)OR^{e}OR^{f}, S(O)R^{b}, S(O)NR^{c}R^{d}, S(O)₂R^{b}, NR^{c}S(O)₂R^{b}, and S(O)₂NR^{c}R^{d};
R is H, C(O)R², C(O)OR³, or C(O)NR⁴R⁵;
R¹ is H or C₁₋₄ alkyl;
R² and R³ are independently selected from H, C₁₋₈ alkyl, aryl, heteroaryl, cycloalkyl, heterocycloalkyl, arylalkyl, heteroarylalkyl, cycloalkylalkyl, and heterocycloalkylalkyl, each optionally substituted by 1, 2, or 3 substituents independently selected from halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, and C₂₋₆ alkynyl;
R⁴ and R⁵ are independently selected from H, C₁₋₈ alkyl, aryl, heteroaryl, cycloalkyl, heterocycloalkyl, arylalkyl, heteroarylalkyl, cycloalkylalkyl, and heterocycloalkylalkyl, each optionally substituted by 1, 2, or 3 substituents independently selected from halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, and C₂₋₆ alkynyl; or
R⁴ and R⁵ together with the N atom to which they are attached form a 4-, 5-, 6- or 7-membered heterocycloalkyl group;
R^{x} is independently selected from H, halo, C₁₋₁₀ alkyl, C₂₋₆ alkenyl, C₂₋₆ alkynyl, pentahalosulfanyl, Cy¹, Cy¹-(C₁₋₆ alkyl), CN, NO₂, OR², SR², C(O)R^{b2}, C(O)NR^{c2}R^{d2}, C(O)OR^{a2}, OC(O)R^{b2}, OC(O)NR^{c2}R^{d2}NR^{c2}R^{d2}NR^{c2}C(O)R^{b2}, NR^{c2}C(O)OR^{a2}, S(O)R^{b2}, S(O)NR^{c2}R^{d2}, S(O)₂R^{b2}NR^{c2}S(O)₂R^{b2} and S(O)₂NR^{c2}R^{d2}; wherein said C₁₋₁₀ alkyl, C₂₋₆ alkenyl, or C₂₋₆ alkynyl is optionally substituted with 1, 2, 3, 4, or 5 substituents independently selected from halo, CN, NO₂, Cy¹, Cy¹-(C₁₋₆ alkyl)-, OR^{a2}, SR^{a2}, C(O)R^{b2}, C(O)NR^{c2}R^{d2}, C(O)OR^{a2}, OC(O)R^{b2}, OC(O)NR^{c2}R^{d2}NR^{c2}R^{d2}, NR^{c2}C(O)R^{b2}, NR^{c2}C(O)OR^{a2}, C(═NRⁱ)NR^{c2}R^{d2}, NR^{c2}C(═NRⁱ)NR^{c2}R^{d2}P(R²)₂, P(OR^{b2})₂, P(O)R^{e2}R^{f2}, P(O)OR^{e2}OR^{f2}, S(O)R^{b2}, S(O)NR^{c2}R^{d2}, S(O)₂R^{b2}, NR^{c2}S(O)₂R^{b2}, and S(O)₂NR^{c2}R^{d2};
Cy and Cy¹ are independently selected from aryl, heteroaryl, cycloalkyl, and heterocycloalkyl, each optionally substituted by 1, 2, 3, 4 or 5 substituents independently selected from halo, C₁₋₄ alkyl, C₂₋₄ alkenyl, C₂₋₄ alkynyl, C₁₋₄ haloalkyl, pentahalosulfanyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, C(═NRⁱ)NR^{c1}R^{d1}, NR^{c1}C(═NRⁱ) NR^{c1}R^{d1}, P(R^{f1})₂, P(OR^{e1})₂, P(O)R^{e1}R^{f1}, P(O)OR^{e1}OR^{f1}, S(O)R^{b1}, S(O)NR^{c1}R^{d1}, S(O)₂R^{b1}, and S(O)₂NR^{c1}R^{d1};
R^{a}, R^{a1} and R^{a2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR)₂, P(O)R′R′, P(O)OR′OR′, S(O)R′, S(O)NR′R′, S(O)₂R′, NR′S(O)₂R′, and S(O)₂NR′R′;
R^{b}, R^{b1} and R^{b2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR)₂, P(O)R′R′, P(O)OR¹⁰R′, S(O)R′, S(O)NR′R′, S(O)₂R′, NR′S(O)₂R′, and S(O)₂NR′R′;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′; or
R^{c}C and R^{d} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
R^{c1} and R^{d1} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′; or
R^{c1} and R^{d1} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
R^{c2} and R^{d2} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
or R^{c2} and R^{d2} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
R^{e}, R^{e1} and R^{b2} are independently selected from H, C₁₋₆ alkyl, C₁₋₆ haloalkyl, C₂₋₆ alkenyl, (C₁₋₆ alkoxy)-C₁₋₆ alkyl, C₂₋₆ alkynyl, aryl, cycloalkyl, heteroaryl, heterocycloalkyl, arylalkyl, cycloalkylalkyl, heteroarylalkyl, and heterocycloalkylalkyl;
R^{f}, R^{f1} and R^{f2} are independently selected from H, C₁₋₆ alkyl, C₁₋₆ haloalkyl, C₂₋₆ alkenyl, C₂₋₆ alkynyl, aryl, cycloalkyl, heteroaryl, and heterocycloalkyl;
Rⁱ is H, CN, or NO₂;
R^{j} and R^{k} are independently selected from H and C₁₋₆ alkyl;
R′ is H, C₁₋₆ alkyl, C₁₋₆ haloalkyl, C₂₋₆ alkenyl, C₂₋₆ alkynyl, aryl, cycloalkyl, heteroaryl, and heterocycloalkyl;
r is 0 or 1; and
s is 0 or 1; with the provisos:
a) when the ring containing U, V, W, X and Y is pyrimidyl, unsubstituted aryl, or unsubstituted pyridyl, and L is a bond, then A is other than unsubstituted phenyl;
b) when A is a phenyl group substituted by CH₃, and L is a bond, then the ring containing U, V, W, X and Y is other than perfluorophenyl;
c) when the ring containing U, V, W, X and Y is unsubstituted phenyl, or phenyl substituted at the 3-position by CN, NO₂, CF₃, OCF₂CHF₂ or Cl, and L is a bond, then A is other than phenyl substituted at the 3-position by Cl or F or at the 4-position by SO₂NR^{c}R^{d};
d) when the ring containing U, V, W, X and Y is unsubstituted pyridyl, and L is a bond, then A is other than unsubstituted naphthyl or phenyl substituted by F, CF₃ or CH₃;
e) when the ring containing U, V, W, X and Y is pyridyl substituted with a CF₃ group, and L is a bond, then A is other than phenyl substituted at the 2-position by C₁ or CH₃, or phenyl substituted at the 3-position by C₁, CF₃ or O-heteroaryl; and
f) when the ring containing U, V, W, X and Y is phenyl substituted with at least one F, Cl, Br, CF₃, CN, NO₂, S(O)₂R^{b2}, and S(O)₂NR^{c2}R^{d2}, C(O)OR^{a2}, Cy¹, OR^{a2}, SR^{a2}, C₁₋₁₀ alkyl, C₂₋₆ alkenyl, or C₂₋₆ alkynyl, and L is a bond; then A is optionally substituted cycloalkyl, optionally substituted heteroaryl, optionally substituted heterocycloalkyl, or aryl substituted by at least two substituents.

2. The compound of claim 1, or pharmaceutically acceptable salt thereof,
wherein U, V, W, X and Y are CR^{x}.

3. The compound of claim 1, or pharmaceutically acceptable salt thereof,
wherein U is N.

4. The compound of claim 2, or pharmaceutically acceptable salt thereof,
wherein X is N.

5. The compound of claim 2, or pharmaceutically acceptable salt thereof,
wherein W is N.

6. The compound of claim 5, or pharmaceutically acceptable salt thereof,
wherein Y is N.

7. The compound of claim 1, or pharmaceutically acceptable salt thereof, wherein V is N.

8. The compound of claim 7, or pharmaceutically acceptable salt thereof, wherein X is N

9. The compound of claim 1, or pharmaceutically acceptable salt thereof, wherein W is N.

10. The compound of claim 1, or pharmaceutically acceptable salt thereof, wherein U and V are both N.

11. The compound of claim 1, or pharmaceutically acceptable salt thereof, wherein A is aryl or heteroaryl, each optionally substituted by 1, 2, 3, 4, or 5 substituents selected from halo, C₁₋₆ alkyl, C₂₋₆ alkenyl, C₂₋₆ alkynyl, C₁₋₆ haloalkyl, C₁₋₆ hydroxyalkyl, C₁₋₆ cyanoalkyl, Cy, CN, NO₂, OR^{a}, SR^{a}, C(O)R^{b}, C(O)NR^{c}R^{d}, C(O)OR^{a}, OC(O)R^{b}, OC(O)NR^{c}R^{d}NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, P(R^{f})₂, P(OR^{e})₂, P(O)R^{e}R^{f}, P(O)OR^{e}OR^{f}, S(O)R^{b}, S(O)NR^{c}R^{d}, S(O)₂R^{b}, NR^{c}S(O)₂R^{b}, and S(O)₂NR^{c}R^{d}, wherein said C₁₋₆ alkyl, C₂₋₆ alkenyl, and C₂₋₆ alkynyl is optionally substituted with 1, 2, or 3 substitutents selected from Cy, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}NR^{c}C(O)OR^{a}, C(═NRⁱ)NR^{c}R^{d}, NR^{c}C(═NRⁱ)NR^{c}R^{d}. P(R^{f})₂, P(OR^{e})₂, P(O)R^{e}R^{f}, P(O)OR^{e}OR^{f}S(O)R^{e}, S(O)NR^{c}R^{d}, S(O)₂R^{b}, NR^{c}S(O)₂R^{b}, and S(O)₂NR^{c}R^{d}.

12. The compound of claim 1, or pharmaceutically acceptable salt thereof, wherein A is phenyl optionally substituted by 1, 2, 3, 4, or 5 substituents selected from halo, C₁₋₆ alkyl, C₂₋₆ alkenyl, C₂₋₆ alkynyl, C₁₋₆ haloalkyl, C₁₋₆ hydroxyalkyl, C₁₋₆ cyanoalkyl, Cy, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}. NR^{c}C(═NRⁱ)NR^{c}R^{d}, P(R^{f})₂, P(OR^{e})₂, P(O)R^{e}R^{f}, P(O)OR^{e}OR^{f}, S(O)R^{e}, S(O)NR^{c}R^{d}, S(O)₂R^{b}, NR^{c}S(O)₂R^{b}, and S(O)₂NR^{c}R^{d}, wherein said C₁₋₆ alkyl, C₂₋₆ alkenyl, and C₂₋₆ alkynyl is optionally substituted with 1, 2, or 3 substitutents selected from Cy, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)O^{a}, C(═NRⁱ)NR^{c}R^{d}, NR^{c}C(═NRⁱ)NR^{c}R^{d}, P(R^{f})₂, P(OR^{e})₂, P(O)R^{e}R^{f}P(O)OR^{e}OR^{f}, S(O)R^{b}S(O)NR^{c}R^{d}, S(O)₂R^{b}, NR^{c}S(O)₂R^{e}, and S(O)₂NR^{c}R^{d}.

13. The compound of claim 1, or pharmaceutically acceptable salt thereof, wherein A is phenyl optionally substituted by 1, 2, 3, 4, or 5 substituents selected from halo, C₁₋₆ alkyl, C₁₋₆ haloalkyl, C₁₋₆ hydroxyalkyl, C₁₋₆ cyanoalkyl, CN, NO₂, OR^{a}, SR^{a}, C(O)R^{b}, C(O)NR^{c}R^{d}, C(O)OR^{a}, OC(O)R^{e}, OC(O)NR^{c}R^{d}, NR^{c}R^{d}, NR^{c}C(O)R^{b}, NR^{c}C(O)NR^{c}R^{d}, NR^{c}C(O)OR^{a}, S(O)R^{b}, S(O)NR^{c}R^{d}, S(O)₂R^{b}, NR^{c}S(O)₂R^{b}, and S(O)₂NR^{c}R^{d}.

14. The compound of claim 1, or pharmaceutically acceptable salt thereof, wherein A is phenyl optionally substituted by 1, 2, 3, 4, or 5 substituents selected from halo, C₁₋₆ alkyl and C₁₋₆ haloalkyl.

15. The compound of claim 1, or pharmaceutically acceptable salt thereof, wherein R¹ is H.

16. The compound of claim 1, or pharmaceutically acceptable salt thereof, wherein R is H.

17. The compound of claim 1, or pharmaceutically acceptable salt thereof, wherein L is a bond, C₁₋₆ alkylene, (C₁₋₆ alkylene)ᵣ-CO—(C₁₋₆ alkylene)ₛ, C₁₋₆ alkylene)ᵣ-NR^{j}—(C₁₋₆ alkylene)ₛ, or (C₁₋₆ alkylene)ᵣ-SO₂—(C₁₋₆ alkylene)ₛ.

18. The compound of claim 1, or pharmaceutically acceptable salt thereof, wherein L is a bond or C₁₋₆ alkylene.

19. The compound of claim 1 having Formula (I): 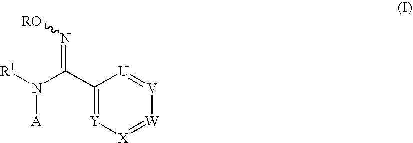
or pharmaceutically acceptable salt thereof.

20. The compound of claim 1 having Formula IIIa or IVa: 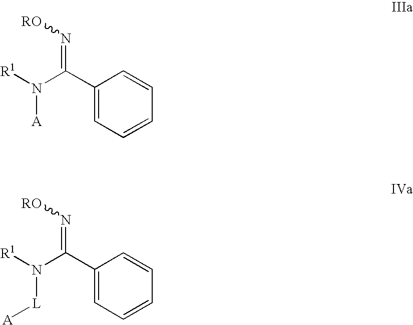
or pharmaceutically acceptable salt thereof.

21. The compound of claim 1 having Formula IIIb or IVb: 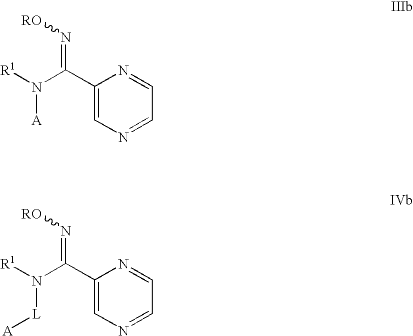
or pharmaceutically acceptable salt thereof.

22. The compound of claim 1 having Formula IIIc or IVc: 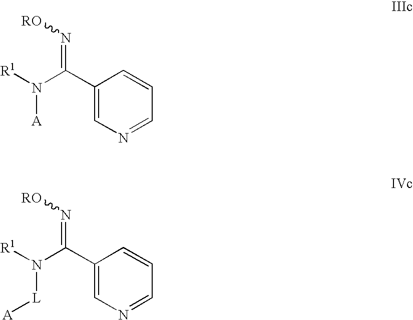
or pharmaceutically acceptable salt thereof.

23. The compound of claim 1 having Formula IIId or IVd: 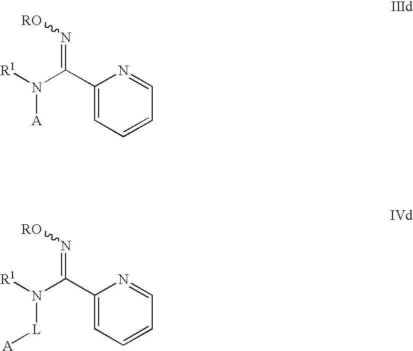
or pharmaceutically acceptable salt thereof.

24. The compound of claim 1 having Formula IIIe or IVe: 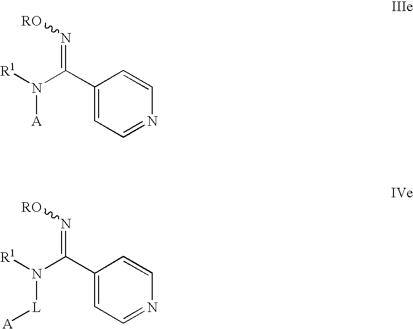
or pharmaceutically acceptable salt thereof.

25. The compound of claim 1 having Formula IIIf or IVf: 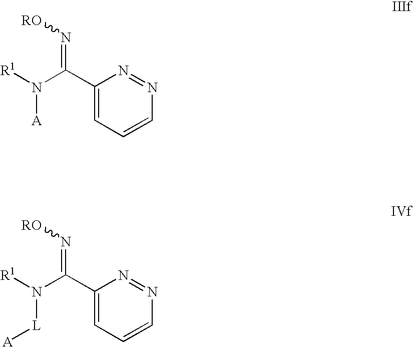
or pharmaceutically acceptable salt thereof.

26. The compound of claim 1 selected from:
N′-hydroxy-3-[(4-methoxybenzyl)amino]-N-[3-(trifluoromethyl)phenyl]pyrazine-2-carboximidamide;
3-amino-N′-hydroxy-N-[3-(trifluoromethyl)phenyl]pyrazine-2-carboximidamide;
N-(3-chloro-4-fluorophenyl)-N′-hydroxybiphenyl-4-carboximidamide;
3-amino-N′-hydroxy-N-[3-(trifluoromethyl)-4-fluorophenyl]pyrazine-2-carboximidamide;
N′-hydroxy-3-[(4-methoxybenzyl)amino]-N-[3-(trifluoromethyl)-4-fluorophenyl]-pyrazine-2-carboximidamide;
3-amino-N′-hydroxy-N-[3-chloro, 4-fluorophenyl]pyrazine-2-carboximidamide;
N′-hydroxy-N-[3-(trifluoromethyl)phenyl]pyrazine-2-carboximidamide;
N′-hydroxy-N-[3-(trifluoromethyl)phenyl]pyridine-3-carboximidamide;
4-(benzyloxy)-N-(3-chloro-4-fluorophenyl)-N′-hydroxybenzenecarboximidamide;
N-(3-chloro-4-fluorophenyl)-N′-hydroxy-3-phenoxybenzenecarboximidamide;
N-(3-chloro-4-fluorophenyl)-N′-hydroxy-3-methoxybenzenecarboximidamide;
N-(3-chloro-4-fluorophenyl)-N′-hydroxy-4-methoxybenzenecarboximidamide;
N-(3-chloro-4-fluorophenyl)-N′-hydroxycyclohexanecarboximidamide;
N-(5-chloro-2-methylbenzyl)-N′-hydroxypyridazine-3-carboximidamide; and
pharmaceutically acceptable salts thereof.

27. A composition comprising a compound of claim 1, or pharmaceutically acceptable salt thereof, and at least one pharmaceutically acceptable carrier.

28. A method of modulating activity of indoleamine 2,3-dioxygenase, comprising contacting said indoleamine 2,3-dioxygenase with a compound of Formula Ia: 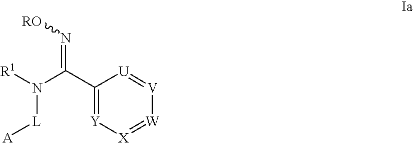
or pharmaceutically acceptable salt thereof, wherein:
U, V, W, X and Y are independently selected from N and CR^{x}, wherein 0, 1, 2 or 3 of U, V, W, X and Y are N, and the remainder of U, V, W, X and Y are CR^{x};
L is a bond, C₁₋₆ alkylene, C₂₋₆ alkenylene, C₂₋₆ alkynylene, (C₁₋₆ alkylene)ᵣ-O—(C₁₋₆ alkylene)ₛ, (C₁₋₆ alkylene)ᵣ-S—(C₁₋₆ alkylene)ₛ, (C₁₋₆ alkylene)ᵣ-NR^{j}—(C₁₋₆ alkylene)ₛ, (C₁₋₆ alkylene)ᵣ-CO—(C₁₋₆ alkylene)ₛ, (C₁₋₆ alkylene)ᵣ-COO—(C₁₋₆ alkylene)ₛ, (C₁₋₆ alkylene)ᵣ-CONR^{j}—(C₁₋₆ alkylene)ₛ, (C₁₋₆ alkylene)ᵣ-SO—(C₁₋₆ alkylene)ₛ, (C₁₋₆ alkylene)ᵣ-SO₂—(C₁₋₆ alkylene)ₛ, (C₁₋₆ alkylene), —SONR^{j}—(C₁₋₆ alkylene)ₛ, (C₁₋₆ alkylene), —SO₂NR^{j}—(C₁₋₆ alkylene)ₛ, or (C₁₋₆ alkylene)ᵣ-NR^{j}CONR^{k}—(C₁₋₆ alkylene)ₛ, wherein each of the C₁₋₆ alkylene, C₂₋₆ alkenylene, and C₂₋₆ alkynylene is optionally substituted by 1, 2, or 3 substituents independently selected from halo, CN, NO₂, N₃, SCN, OH, C₁₋₆ alkyl, C₁₋₆ haloalkyl, C₂₋₈ alkoxyalkyl, C₁₋₆ alkoxy, C₁₋₆ haloalkoxy, amino, C₁₋₆ alkylamino, and C₂₋₈ dialkylamino;
A is aryl, cycloalkyl, heteroaryl, or heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₁₋₆ hydroxyalkyl, C₁₋₆ cyanoalkyl, pentahalosulfanyl, Cy, CN, ORE, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}., P(R^{f})₂, P(OR)₂, P(O)R^{e}R^{f}, P(O)OR^{e}OR^{f}, S(O)R^{e}, S(O)NR^{c}R^{d}, S(O)₂R^{b}, NR^{c}S(O)₂R^{b}, and S(O)₂NR^{c}R^{d}, wherein said C₁₋₆ alkyl, C₂₋₆ alkenyl, and C₂₋₆ alkynyl is optionally substituted with 1, 2, or 3 substituents independently selected from Cy, CN, NO₂, OR^{a}, SR^{a}, C(O)R^{b}, C(O)NR^{c}R^{d}, C(O)OR^{a}, OC(O)R^{b}, OC(O)NR^{c}R^{d}., NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, P(R^{f})₂, P(OR^{e})₂, P(O)R^{e}R^{f}P(O)OR^{e}OR^{f}S(O)R^{b}S(O)NR^{c}R^{d}, S(O)₂R^{b}, NR^{c}S(O)₂R^{b}, and S(O)₂NR^{c}R^{d};
R is H, C(O)R², C(O)OR³, or C(O)NR⁴R⁵;
R¹ is H or C₁₋₄ alkyl;
R² and R³ are independently selected from H, C₁₋₈ alkyl, aryl, heteroaryl, cycloalkyl, heterocycloalkyl, arylalkyl, heteroarylalkyl, cycloalkylalkyl, and heterocycloalkylalkyl, each optionally substituted by 1, 2, or 3 substituents independently selected from halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, and C₂₋₆ alkynyl;
R⁴ and R⁵ are independently selected from H, C₁₋₈ alkyl, aryl, heteroaryl, cycloalkyl, heterocycloalkyl, arylalkyl, heteroarylalkyl, cycloalkylalkyl, and heterocycloalkylalkyl, each optionally substituted by 1, 2, or 3 substituents independently selected from halo, CN, NO₂, OH, C₁₋₄ alkoxy, C₁₋₄ haloalkoxy, amino, C₁₋₄ alkylamino, C2-s dialkylamino, C₁₋₆ alkyl, C₂₋₆ alkenyl, and C₂₋₆ alkynyl; or
R⁴ and R⁵ together with the N atom to which they are attached form a 4-, 5-, 6- or 7-membered heterocycloalkyl group;
R^{x} is independently selected from H, halo, C₁₋₁₀ alkyl, C₂₋₆ alkenyl, C₂₋₆ alkynyl, pentahalosulfanyl, Cy¹, Cy¹-(C₁₋₆ alkyl), CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, S(O)R^{b2}, S(O)NR^{c2}R^{d2}, S(O)₂R^{b2}, NR^{c2}S(O)₂R^{b2}, and S(O)₂NR^{c2}R²; wherein said C₁₋₁₀ alkyl, C₂₋₆ alkenyl, or C₂₋₆ alkynyl is optionally substituted with 1, 2, 3, 4, or 5 substituents independently selected from halo, CN, NO₂, Cy¹, Cy¹-(C₁₋₆ alkyl)-, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}NR^{c2}C(O)R^{b2}, NR^{c2}C(O)OR^{a2}, C(═NRⁱ)NR^{c2}R^{d2}, NR^{c2}C(═NRⁱ)NR^{c2}R^{d2}P(R²)₂, p(OR^{b2})₂, P(O)R^{e2}R^{f2}, P(O)OR^{e2}OR^{f2}, S(O)R^{b2}S(O)NR^{c2}R^{d2}, S(O)₂R² and NR^{c2}S(O)₂R^{b2}, S(O)₂NR^{c2}R^{d2};
Cy and Cy¹ are independently selected from aryl, heteroaryl, cycloalkyl, and heterocycloalkyl, each optionally substituted by 1, 2, 3, 4 or 5 substituents independently selected from halo, C₁₋₄ alkyl, C₂₋₄ alkenyl, C₂₋₄ alkynyl, C₁₋₄ haloalkyl, pentahalosulfanyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, C(═NRⁱ)NR^{c1}R^{d1}, NR^{c1}C(═NRⁱ)NR^{c1}R^{d1}, P(R^{f1})₂, P(OR^{e1})₂, P(O)R^{e1}R^{f1}, P(O)OR^{e1}OR^{f1}, S(O)R^{b1}, S(O)NR^{c1}R^{d1}, S(O)₂R^{b1}, and S(O)₂NR^{c1}R^{d1}
R^{a}, R^{a1} and R^{a2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′OR′, S(O)R′, S(O)NR′R′, S(O)₂R′, NR′S(O)₂R′, and S(O)₂NR′R′;
R^{b}, R^{b1} and R^{b2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′OR′, S(O)R′, S(O)NR′R′, S(O)₂R′, NR′S(O)₂R′, and S(O)₂NR′R′;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′; or
R^{c} and R^{d} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
R^{c1} and R^{d1} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′; or
R^{c1} and R^{d1} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
R^{c2} and R^{d2} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
or R^{c2} and R^{d2} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
R^{e}, R^{e1} and R^{b2} are independently selected from H, C₁₋₆ alkyl, C₁₋₆ haloalkyl, C₂₋₆ alkenyl, (C₁₋₆ alkoxy)-C₁₋₆ alkyl, C₂₋₆ alkynyl, aryl, cycloalkyl, heteroaryl, heterocycloalkyl, arylalkyl, cycloalkylalkyl, heteroarylalkyl, and heterocycloalkylalkyl;
R^{f}, R^{f1} and R^{f2} are independently selected from H, C₁₋₆ alkyl, C₁₋₆ haloalkyl, C₂₋₆ alkenyl, C₂₋₆ alkynyl, aryl, cycloalkyl, heteroaryl, and heterocycloalkyl;
Rⁱ is H, CN, or NO₂;
R^{j} and R^{k} are independently selected from H and C₁₋₆ alkyl;
R′ is H, C₁₋₆ alkyl, C₁₋₆ haloalkyl, C₂₋₆ alkenyl, C₂₋₆ alkynyl, aryl, cycloalkyl, heteroaryl, and heterocycloalkyl;
r is 0 or 1; and
s is 0 or 1.

29. The method of claim 28 wherein said modulating is inhibiting.

30. A method of inhibiting immunosuppression in a patient, comprising administering to said patient an effective amount of a compound of Formula Ia: 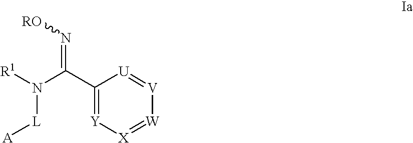
or pharmaceutically acceptable salt thereof, wherein:
U, V, W, X and Y are independently selected from N and CR^{x}, wherein 0, 1, 2 or 3 of U, V, W, X and Y are N, and the remainder of U, V, W, X and Y are CR^{x};
L is a bond, C₁₋₆ alkylene, C₂₋₆ alkenylene, C₂₋₆ alkynylene, (C₁₋₆ alkylene)ᵣ-O—(C₁₋₆ alkylene)ₛ, (C₁₋₆ alkylene)ᵣ-S—(C₁₋₆ alkylene)ₛ, (C₁₋₆ alkylene), —NR^{j}—(C₁₋₆ alkylene)ₛ, (C₁₋₆ alkylene)ᵣ-CO—(C₁₋₆ alkylene)ₛ, (C₁₋₆ alkylene)ᵣ-COO—(C₁₋₆ alkylene)ₛ, (C₁₋₆ alkylene)ᵣ-CONR^{j}—(C₁₋₆ alkylene)ₛ, (C₁₋₆ alkylene)ᵣ-SO—(C₁₋₆ alkylene)ₛ, (C₁₋₆ alkylene)ᵣ-SO₂—(C₁₋₆ alkylene)ₛ, (C₁₋₆ alkylene)ᵣ-SONR^{j}—(C₁₋₆ alkylene)ₛ, or (C₁₋₆ alkylene)ᵣ-NR^{j}CONR^{k}—(C₁₋₆ alkylene)ₛ, wherein each of the C₁₋₆ alkylene, C₂₋₆ alkenylene, and C₂₋₆ alkynylene is optionally substituted by 1, 2, or 3 substituents independently selected from halo, CN, NO₂, N₃, SCN, OH, C₁₋₆ alkyl, C₁₋₆ haloalkyl, C₂₋₈ alkoxyalkyl, C₁₋₆ alkoxy, C₁₋₆ haloalkoxy, amino, C₁₋₆ alkylamino, and C₂₋₈ dialkylamino;
A is aryl, cycloalkyl, heteroaryl, or heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₁₋₆ hydroxyalkyl, C₁₋₆ cyanoalkyl, pentahalosulfanyl, Cy, CN, ORE, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}., P(R^{f})₂, P(OR)₂, P(O)R^{e}R^{f}, P(O)OR^{e}OR^{f}, S(O)R^{e}, S(O)NR^{c}R^{d}, S(O)₂R^{b}, NR^{c}S(O)₂R^{b}, and S(O)₂NR^{c}R^{d}, wherein said C₁₋₆ alkyl, C₂₋₆ alkenyl, and C₂₋₆ alkynyl is optionally substituted with 1, 2, or 3 substituents independently selected from Cy, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, P(R^{f})₂, P(OR^{e})₂, P(O)R^{e}R^{f}P(O)OR^{e}OR^{f}S(O)R^{b}S(O)NR^{c}R^{d}, S(O)₂R^{b}, NR^{c}S(O)₂R^{b}, and S(O)₂NR^{c}R^{d};
R is H, C(O)R², C(O)OR³, or C(O)NR⁴R⁵;
R¹ is H or C₁₋₄ alkyl;
R² and R³ are independently selected from H, C₁₋₈ alkyl, aryl, heteroaryl, cycloalkyl, heterocycloalkyl, arylalkyl, heteroarylalkyl, cycloalkylalkyl, and heterocycloalkylalkyl, each optionally substituted by 1, 2, or 3 substituents independently selected from halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, and C₂₋₆ alkynyl;
R⁴ and R⁵ are independently selected from H, C₁₋₈ alkyl, aryl, heteroaryl, cycloalkyl, heterocycloalkyl, arylalkyl, heteroarylalkyl, cycloalkylalkyl, and heterocycloalkylalkyl, each optionally substituted by 1, 2, or 3 substituents independently selected from halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, and C₂₋₆ alkynyl; or
R⁴ and R⁵ together with the N atom to which they are attached form a 4-, 5-, 6- or 7-membered heterocycloalkyl group;
R^{x} is independently selected from H, halo, C₁₋₁₀ alkyl, C₂₋₆ alkenyl, C₂₋₆ alkynyl, pentahalosulfanyl, Cy¹, Cy¹-(C₁₋₆ alkyl), CN, NO₂, OR², SR², C(O)R^{b2}, C(O)NR^{c2}R^{d2}, C(O)OR^{a2}, OC(O)R^{b2}, OC(O)NR^{c2}R^{d2}NR^{c2}R^{d2}NR^{c2}C(O)R^{b2}, NR^{c2}C(O)OR^{a2}, S(O)R^{b2}, S(O)NR^{c2}R^{d2}, S(O)₂R^{b2}, NR^{c2}S(O)₂R² and S(O)₂NR^{c2}R^{d2}; wherein said C₁₋₁₀ alkyl, C₂₋₆ alkenyl, or C₂₋₆ alkynyl is optionally substituted with 1, 2, 3, 4, or 5 substituents independently selected from halo, CN, NO₂, Cy¹, Cy¹-(C₁₋₆ alkyl)-, OR^{a2}, SR^{a2}, C(O)R^{b2}, C(O)NR^{c2}R^{d2}, C(O)OR^{a2}, OC(O)R²OC(O)NR^{c2}R^{d2}, NR^{c2}R^{d2}, NR^{c2}C(O)R^{b2}, NR^{c2}C(O)OR^{a2}C(═NRⁱ)NR^{c2}R^{d2}, NR^{c2}C(═NRⁱ)NR^{c2}R^{d2}P(R^{e2})₂, p(OR^{e2})₂, P(O)R^{e2}R^{f2}, P(O)OR^{e2}OR^{f2}, S(O)R^{b2}, S(O)NR^{c2}R^{d2}S(O)₂R²NR^{c2}S(O)₂R^{b2}, and S(O)₂NR^{c2}R^{d2};
Cy and Cy¹ are independently selected from aryl, heteroaryl, cycloalkyl, and heterocycloalkyl, each optionally substituted by 1, 2, 3, 4 or 5 substituents independently selected from halo, C₁₋₄ alkyl, C₂₋₄ alkenyl, C₂₋₄ alkynyl, C₁₋₄ haloalkyl, pentahalosulfanyl, CN, NO₂, OR^{a1}, SR^{a1}, C(O)R^{b1}, C(O)NR^{c1}R^{d2}, C(O)OR^{a1}, OC(O)R^{b1}, OC(O)NR^{c1}R^{d1}, NR^{c1}R^{d}, NR^{c1}C(O)R^{b1}, NR^{c1}C(O)OR^{a1}, C(═NRⁱ)NR^{c1}R^{d1}, NR^{c1}C(═NRⁱ)NR^{c1}R^{d1}, P(R^{f1})₂, P(OR^{e1})₂, P(O)R^{e1}R^{f1}, P(O)OR^{e1}OR^{f1}, S(O)R^{b1}, S(O)NR^{c1}R^{d2}, S(O)₂R^{e1}, and S(O)₂NR^{c1}R^{d1};
R^{a}, R^{a1} and R^{a2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′OR′, S(O)R′, S(O)NR′R′, S(O)₂R′, NR′S(O)₂R′, and S(O)₂NR′R′;
R^{b}, R^{b1} and R^{b2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′OR′, S(O)R′, S(O)NR′R′, S(O)₂R′, NR′S(O)₂R′, and S(O)₂NR′R′;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′; or
R^{c} and R^{d} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
R^{c1} and R^{d1} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′; or
R^{c2} and R^{d2} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
R^{c2} and R^{d2} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
or R^{c2} and R^{d2} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
R^{e}, R^{e1} and R^{e2} are independently selected from H, C₁₋₆ alkyl, C₁₋₆ haloalkyl, C₂₋₆ alkenyl, (C₁₋₆ alkoxy)-C₁₋₆ alkyl, C₂₋₆ alkynyl, aryl, cycloalkyl, heteroaryl, heterocycloalkyl, arylalkyl, cycloalkylalkyl, heteroarylalkyl, and heterocycloalkylalkyl;
R^{f}, R^{f1} and R^{f2} are independently selected from H, C₁₋₆ alkyl, C₁₋₆ haloalkyl, C₂₋₆ alkenyl, C₂₋₆ alkynyl, aryl, cycloalkyl, heteroaryl, and heterocycloalkyl;
Rⁱ is H, CN, or NO₂;
R^{j} and R^{k} are independently selected from H and C₁₋₆ alkyl;
R′ is H, C₁₋₆ alkyl, C₁₋₆ haloalkyl, C₂₋₆ alkenyl, C₂₋₆ alkynyl, aryl, cycloalkyl, heteroaryl, and heterocycloalkyl;
r is 0 or 1; and
s is 0 or 1.

31. A method of treating cancer, viral infection, depression, a neurodegenerative disorder, trauma, age-related cataracts, organ transplant rejection, or an autoimmune disease in a patient, said method comprising administering to said patient a therapeutically effective amount of a compound of Formula Ia: 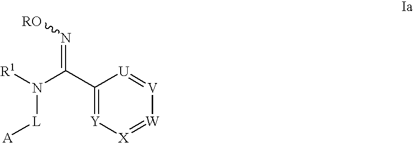
or pharmaceutically acceptable salt thereof, wherein:
U, V, W, X and Y are independently selected from N and CR^{x}, wherein 0, 1, 2 or 3 of U, V, W, X and Y are N, and the remainder of U, V, W, X and Y are CR^{x};
L is a bond, C₁₋₆ alkylene, C₂₋₆ alkenylene, C₂₋₆ alkynylene, (C₁₋₆ alkylene)ᵣ-O—(C₁₋₆ alkylene)ₛ, (C₁₋₆ alkylene)ᵣ-S—(C₁₋₆ alkylene)ₛ, (C₁₋₆ alkylene)ᵣ-NR^{j}—(C₁₋₆ alkylene)ₛ, (C₁₋₆ alkylene)ᵣ-CO—(C₁₋₆ alkylene)ₛ, (C₁₋₆ alkylene)ᵣ-COO—(C₁₋₆ alkylene)ₛ, (C₁₋₆ alkylene)ᵣ-CONR^{j}—(C₁₋₆ alkylene)ₛ, (C₁₋₆ alkylene)ᵣ-SO—(C₁₋₆ alkylene)ₛ, (C₁₋₆ alkylene)ᵣ-SO₂—(C₁₋₆ alkylene)ₛ, (C₁₋₆ alkylene)ᵣ-SONR^{j}—(C₁₋₆ alkylene)ₛ, or (C₁₋₆ alkylene)ᵣ-NR^{j}CONR^{k}—(C₁₋₆ alkylene)ₛ, wherein each of the C₁₋₆ alkylene, C₂₋₆ alkenylene, and C₂₋₆ alkynylene is optionally substituted by 1, 2, or 3 substituents independently selected from halo, CN, NO₂, N₃, SCN, OH, C₁₋₆ alkyl, C₁₋₆ haloalkyl, C₂₋₈ alkoxyalkyl, C₁₋₆ alkoxy, C₁₋₆ haloalkoxy, amino, C₁₋₆ alkylamino, and C₂₋₈ dialkylamino;
A is aryl, cycloalkyl, heteroaryl, or heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₁₋₆ hydroxyalkyl, C₁₋₆ cyanoalkyl, pentahalosulfanyl, Cy, CN, ORE, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}C(═NRⁱ)NR^{c}R^{d}, NR^{c}C(═NRⁱ)NR^{c}R^{d}, P(R^{f})₂, P(OR)₂, P(O)R^{e}R^{f}, P(O)OR^{e}OR^{f}, S(O)R^{e}, S(O)NR^{c}R^{d}, S(O)₂R^{b}, NR^{c}S(O)₂R^{b}, and S(O)₂NR^{c}R^{d}, wherein said C₁₋₆ alkyl, C₂₋₆ alkenyl, and C₂₋₆ alkynyl is optionally substituted with 1, 2, or 3 substituents independently selected from Cy, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, P(R^{f})₂, P(OR^{e})₂, P(O)R^{e}R^{f}P(O)OR^{e}OR^{f}S(O)R^{b}S(O)NR^{c}R^{d}, S(O)₂R^{b}, NR^{c}S(O)₂R^{b}, and S(O)₂NR^{c}R^{d};
R is H, C(O)R², C(O)OR³, or C(O)NR⁴R⁵;
R¹ is H or C₁₋₄ alkyl;
R² and R³ are independently selected from H, C₁₋₈ alkyl, aryl, heteroaryl, cycloalkyl, heterocycloalkyl, arylalkyl, heteroarylalkyl, cycloalkylalkyl, and heterocycloalkylalkyl, each optionally substituted by 1, 2, or 3 substituents independently selected from halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, and C₂₋₆ alkynyl;
R⁴ and R⁵ are independently selected from H, C₁₋₈ alkyl, aryl, heteroaryl, cycloalkyl, heterocycloalkyl, arylalkyl, heteroarylalkyl, cycloalkylalkyl, and heterocycloalkylalkyl, each optionally substituted by 1, 2, or 3 substituents independently selected from halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, and C₂₋₆ alkynyl; or
R⁴ and R⁵ together with the N atom to which they are attached form a 4-, 5-, 6- or 7-membered heterocycloalkyl group;
R^{x} is independently selected from H, halo, C₁₋₁₀ alkyl, C₂₋₆ alkenyl, C₂₋₆ alkynyl, pentahalosulfanyl, Cy¹, Cy¹-(C₁₋₆ alkyl), CN, NO₂, OR¹², SR¹², C(O)R^{b2}, C(O)NR^{c2}R^{d2}, C(O)OR^{a2}, OC(O)R^{b2}OC(O)NR^{c2}R^{d2}, NR^{c2}R^{d2}, NR^{c2}C(O)R^{b2}, NR^{c2}C(O)OR^{a2}, S(O)R^{b2}, S(O)NR^{c2}R^{d2}, S(O)₂R^{b2}NR^{c2}S(O)₂R^{b2} and S(O)₂NR^{c2}R^{d2}; wherein said C₁₋₁₀ alkyl, C₂₋₆ alkenyl, or C₂₋₆ alkynyl is optionally substituted with 1, 2, 3, 4, or 5 substituents independently selected from halo, CN, NO₂, Cy¹, Cy¹-(C₁₋₆ alkyl)-, OR^{a2}, SR^{a2}, C(O)R^{b2}, C(O)NR^{c2}R^{d2}, C(O)OR^{a2}, OC(O)R^{b2}, OC(O)NR^{c2}R^{d2}NR^{c2}R^{d2}, NR^{c2}C(O)R^{b2}, NR^{c2}C(O)OR^{a2}, C(═NRⁱ)NR^{c2}R^{d2}, NR^{c2}C(═NRⁱ)NR^{c2}R^{d2}P(R²)₂, p(OR^{b2})₂, P(O)R^{e2}R^{f2}, P(O)OR^{e2}OR^{f2}S(O)R^{b2}, S(O)NR^{c2}R^{d2}, S(O)₂R^{b2}, NR^{c2}S(O)₂R^{b2}, and S(O)₂NR^{c2}R^{d2};
Cy and Cy¹ are independently selected from aryl, heteroaryl, cycloalkyl, and heterocycloalkyl, each optionally substituted by 1, 2, 3, 4 or 5 substituents independently selected from halo, C₁₋₄ alkyl, C₂₋₄ alkenyl, C₂₋₄ alkynyl, C₁₋₄ haloalkyl, pentahalosulfanyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, C(═NRⁱ)NR^{c1}R^{d1}, NR^{c1}C(═NRⁱ)NR^{c1}R^{d1}, P(R^{f1})₂, P(OR^{e1})₂, P(O)R^{e1}R^{f1}, P(O)OR^{e1}OR^{f1}, S(O)R^{b1}, S(O)NR^{c1}R^{d2}, S(O)₂R^{e1}, and S(O)₂NR^{c1}R^{d1} R^{a}, R^{a1} and R^{a2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′OR′, S(O)R′, S(O)NR′R′, S(O)₂R′, NR′S(O)₂R′, and S(O)₂NR′R′;
R^{b}, R^{b1} and R^{b2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR)₂, P(O)R′R′, P(O)OR′OR′, S(O)R′, S(O)NR′R′, S(O)₂R′, NR′S(O)₂R′, and S(O)₂NR′R′;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′; or
R^{c} and R^{d} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
R^{c1} and R^{d1} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′; or
R^{c1} and R^{d1} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
R^{c2} and R^{d2} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
or R^{c2} and R^{d2} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
R^{e}, R^{e1} and R^{b2} are independently selected from H, C₁₋₆ alkyl, C₁₋₆ haloalkyl, C₂₋₆ alkenyl, (C₁₋₆ alkoxy)-C₁₋₆ alkyl, C₂₋₆ alkynyl, aryl, cycloalkyl, heteroaryl, heterocycloalkyl, arylalkyl, cycloalkylalkyl, heteroarylalkyl, and heterocycloalkylalkyl;
R^{f}, R^{f1} and R^{f2} are independently selected from H, C₁₋₆ alkyl, C₁₋₆ haloalkyl, C₂₋₆ alkenyl, C₂₋₆ alkynyl, aryl, cycloalkyl, heteroaryl, and heterocycloalkyl;
Rⁱ is H, CN, or NO₂;
R^{j} and R^{k} are independently selected from H and C₁₋₆ alkyl;
R′ is H, C₁₋₆ alkyl, C₁₋₆ haloalkyl, C₂₋₆ alkenyl, C₂₋₆ alkynyl, aryl, cycloalkyl, heteroaryl, and heterocycloalkyl;
r is 0 or 1;
s is 0 or 1.

32. The method of claim 31 further comprising administering an anti-viral agent, a chemotherapeutic, an immunosuppressant, radiation, an anti-tumor vaccine, an anti-viral vaccine, cytokine therapy, or a tyrosine kinase inhibitor.

33. A method of treating melanoma in a patient, said method comprising administering to said patient a therapeutically effective amount of a compound of Formula Ia: 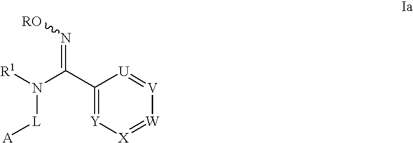
or pharmaceutically acceptable salt, wherein:
U, V, W, X and Y are independently selected from N and CR^{x}, wherein 0, 1, 2 or 3 of U, V, W, X and Y are N, and the remainder of U, V, W, X and Y are CR^{x};
L is a bond, C₁₋₆ alkylene, C₂₋₆ alkenylene, C₂₋₆ alkynylene, (C₁₋₆ alkylene)ᵣ-O—(C₁₋₆ alkylene)ₛ, (C₁₋₆ alkylene)ᵣ-S—(C₁₋₆ alkylene)ₛ, (C₁₋₆ alkylene)ᵣ-NR^{j}—(C₁₋₆ alkylene)ₛ, (C₁₋₆ alkylene)ᵣ-CO—(C₁₋₆ alkylene)ₛ, (C₁₋₆ alkylene)ᵣ-COO—(C₁₋₆ alkylene)ₛ, (C₁₋₆ alkylene)ᵣ-CONR^{j}—(C₁₋₆ alkylene)ₛ, (C₁₋₆ alkylene)ᵣ-SO—(C₁₋₆ alkylene)ₛ, (C₁₋₆ alkylene)ᵣ-SO₂—(C₁₋₆ alkylene)ₛ, (C₁₋₆ alkylene)ᵣ-SONR^{j}—(C₁₋₆ alkylene)ₛ, or (C₁₋₆ alkylene)ᵣ-NR^{j}CONR^{k}—(C₁₋₆ alkylene)ₛ, wherein each of the C₁₋₆ alkylene, C₂₋₆ alkenylene, and C₂₋₆ alkynylene is optionally substituted by 1, 2, or 3 substituents independently selected from halo, CN, NO₂, N₃, SCN, OH, C₁₋₆ alkyl, C₁₋₆ haloalkyl, C₂₋₈ alkoxyalkyl, C₁₋₆ alkoxy, C₁₋₆ haloalkoxy, amino, C₁₋₆ alkylamino, and C₂₋₈ dialkylamino;
A is aryl, cycloalkyl, heteroaryl, or heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₁₋₆ hydroxyalkyl, C₁₋₆ cyanoalkyl, pentahalosulfanyl, Cy, CN, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}C(═NRⁱ)NR^{c}R^{d}, NR^{c}C(═NRⁱ)NR^{c}R^{d}., P(R^{f})₂, P(OR^{e})₂, P(O)R^{e}R^{f}, P(O)OR^{e}OR^{f}S(O)R^{e}, S(O)NR^{c}R^{d}, S(O)₂R^{b}, NR^{c}S(O)₂R^{b}, and S(O)₂NR^{c}R^{d}, wherein said C₁₋₆ alkyl, C₂₋₆ alkenyl, and C₂₋₆ alkynyl is optionally substituted with 1, 2, or 3 substituents independently selected from Cy, CN, NO₂, OR^{a}, SR^{a}, C(O)R^{b}, C(O)NR^{c}R^{d}, C(O)OR^{a}, OC(O)R^{e}, OC(O)NR^{c}R^{d}. NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, P(R^{f})₂, P(OR^{e})₂, P(O)R^{e}R^{f}P(O)OR^{e}OR^{f}, S(O)R^{b}S(O)NR^{c}R^{d}, S(O)₂R^{b}, NR^{c}S(O)₂R^{b}, and S(O)₂NR^{c}R^{d};
R is H, C(O)R², C(O)OR³, or C(O)NR⁴R⁵;
R¹ is H or C₁₋₄ alkyl;
R² and R³ are independently selected from H, C₁₋₈ alkyl, aryl, heteroaryl, cycloalkyl, heterocycloalkyl, arylalkyl, heteroarylalkyl, cycloalkylalkyl, and heterocycloalkylalkyl, each optionally substituted by 1, 2, or 3 substituents independently selected from halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, and C₂₋₆ alkynyl;
R⁴ and R⁵ are independently selected from H, C₁₋₈ alkyl, aryl, heteroaryl, cycloalkyl, heterocycloalkyl, arylalkyl, heteroarylalkyl, cycloalkylalkyl, and heterocycloalkylalkyl, each optionally substituted by 1, 2, or 3 substituents independently selected from halo, CN, NO₂, OH, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, and C₂₋₆ alkynyl; or
R⁴ and R⁵ together with the N atom to which they are attached form a 4-, 5-, 6- or 7-membered heterocycloalkyl group;
R^{x} is independently selected from H, halo, C₁₋₁₀ alkyl, C₂₋₆ alkenyl, C₂₋₆ alkynyl, pentahalosulfanyl, Cy¹, Cy¹-(C₁₋₆ alkyl), CN, NO₂, OR², SR², C(O)R^{b2}, C(O)NR^{c2}R^{d2}, C(O)OR^{a2}, OC(O)R^{b2}, OC(O)NR^{c2}R^{d2}NR^{c2}R^{d2}NR^{c2}C(O)R^{b2}, NR^{c2}C(O)OR^{a2}, S(O)R^{b2}, S(O)NR^{c2}R^{d2}, S(O)₂R^{b2}, NR^{c2}S(O)₂R^{b2} and S(O)₂NR^{c2}R^{d2}; wherein said C₁₋₁₀ alkyl, C₂₋₆ alkenyl, or C₂₋₆ alkynyl is optionally substituted with 1, 2, 3, 4, or 5 substituents independently selected from halo, CN, NO₂, Cy¹, Cy¹-(C₁₋₆ alkyl)-, OR^{a2}, SR^{a2}, C(O)R^{b2}, C(O)NR^{c2}R^{d2}, C(O)OR^{a2}, OC(O)R^{b2}, OC(O)NR^{c2}R^{d2}NR^{c2}R^{d2}, NR^{c2}C(O)R^{b2}, NR_{c2}C(O)OR^{a2}, C(═NRⁱ)NR^{c2}R^{d2}, NR^{c2}C(═NRⁱ)NR^{c2}R^{d2}P(R^{f2})₂, P(OR^{e2})₂, P(O)R^{e2}, R^{f2}, P(O)OR^{e2}OR^{f2}S(O)R^{b2}S(O)NR^{c2}R^{d2}S(O)₂R^{b2}, NR^{c2}S(O)₂R^{b2}, and S(O)₂NR^{c2}R^{d2};
Cy and Cy¹ are independently selected from aryl, heteroaryl, cycloalkyl, and heterocycloalkyl, each optionally substituted by 1, 2, 3, 4 or 5 substituents independently selected from halo, C₁₋₄ alkyl, C₂₋₄ alkenyl, C₂₋₄ alkynyl, C₁₋₄ haloalkyl, pentahalosulfanyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, C(═NRⁱ)NR^{c1}R^{d1}, NR^{c1}C(═NRⁱ)NR^{c1}R^{d1}, P(R^{f1})₂, P(OR^{e1})₂, P(O)R^{e1}R^{f1}, P(O)OR^{e1}OR^{f1}, S(O)R^{b1}, S(O)NR^{c1}R^{d2}, S(O)₂R^{e1}, and S(O)₂NR^{c1}R^{d1};
R^{a}, R^{a1} and R^{a2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′OR′, S(O)R′, S(O)NR′R′, S(O)₂R′, NR′S(O)₂R′, and S(O)₂NR′R′;
R^{b}, R^{b1} and R^{b2} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl is optionally substituted with 1, 2, or 3 substituents independently selected from halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR)₂, P(O)R′R′, P(O)OR¹⁰R′, S(O)R′, S(O)NR′R′, S(O)₂R′, NR′S(O)₂R′, and S(O)₂NR′R′;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′; or
R^{c} and R^{d} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
R^{c1} and R^{d1} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′; or
R^{c1} and R^{d1} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
R^{c2} and R^{d2} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
or R^{c2} and R^{d2} together with the N atom to which they are attached form a 4-, 5-, 6- or 7-membered heterocycloalkyl group optionally substituted with halo, C₁₋₆ alkyl, aryl, arylalkyl, heteroaryl, heteroarylalkyl, cycloalkyl, heterocycloalkyl, CN, NO₂, OR′, SR′, C(O)R′, C(O)NR′R′, C(O)OR′, OC(O)R′, OC(O)NR′R′, NR′R′, NR′C(O)R′, NR′C(O)OR′, C(═NRⁱ)NR′R′, NR′C(═NRⁱ)NR′R′, P(R′)₂, P(OR′)₂, P(O)R′R′, P(O)OR′R′, S(O)R′, S(O)NR′R′, S(O)₂R′, NR′S(O)₂R′, and S(O)₂NR′R′;
R^{e}, R^{e1} and R^{e2} are independently selected from H, C₁₋₆ alkyl, C₁₋₆ haloalkyl, C₂₋₆ alkenyl, (C₁₋₆ alkoxy)-C₁₋₆ alkyl, C₂₋₆ alkynyl, aryl, cycloalkyl, heteroaryl, heterocycloalkyl, arylalkyl, cycloalkylalkyl, heteroarylalkyl, and heterocycloalkylalkyl;
R^{f}, R^{f1} and R^{f2} are independently selected from H, C₁₋₆ alkyl, C₁₋₆ haloalkyl, C₂₋₆ alkenyl, C₂₋₆ alkynyl, aryl, cycloalkyl, heteroaryl, and heterocycloalkyl;
Rⁱ is H, CN, or NO₂;
R^{j} and R^{k} are independently selected from H and C₁₋₆ alkyl;
R′ is H, C₁₋₆ alkyl, C₁₋₆ haloalkyl, C₂₋₆ alkenyl, C₂₋₆ alkynyl, aryl, cycloalkyl, heteroaryl, and heterocycloalkyl;
r is 0 or 1; and
s is 0 or 1.

34. The method of claim 33 further comprising administering an anti-viral agent, a chemotherapeutic, an immunosuppressant, radiation, an anti-tumor vaccine, an anti-viral vaccine, cytokine therapy, or a tyrosine kinase inhibitor.
